SEQUENCE LISTING (39 sequences)

SEQ ID NO: 1 - DNA, 20 bp, synthetic construct
  misc_feature 1..20: note = Synthetic: sgRNA #1 (eCHO1)
  source 1..20: mol_type = other DNA
gccccgagacatgatcacgg

SEQ ID NO: 2 - DNA, 20 bp, synthetic construct
  misc_feature 1..20: note = Synthetic: sgRNA #2 (eCHO2)
  source 1..20: mol_type = other DNA
ccgcctccgtgatcatgtct

SEQ ID NO: 3 - DNA, 20 bp, synthetic construct
  misc_feature 1..20: note = Synthetic: sgRNA #3 (eCHO3)
  source 1..20: mol_type = other DNA
gcctccgtgatcatgtctcg

SEQ ID NO: 4 - DNA, 20 bp, synthetic construct
  misc_feature 1..20: note = Synthetic: sgRNA #4 (eCHO4)
  source 1..20: mol_type = other DNA
cttcggcggaggcaccaagc

SEQ ID NO: 5 - DNA, 20 bp, synthetic construct
  misc_feature 1..20: note = Synthetic: sgRNA #5 (eCHO5)
  source 1..20: mol_type = other DNA
gcaccaagctggaaatcaag

SEQ ID NO: 6 - DNA, 20 bp, synthetic construct
  misc_feature 1..20: note = Synthetic: sgRNA #6 (eCHO6)
  source 1..20: mol_type = other DNA
gaacacggagggagcggcca

SEQ ID NO: 7 - DNA, 20 bp, synthetic construct
  misc_feature 1..20: note = Synthetic: sgRNA #7 (eCHO7)
  source 1..20: mol_type = other DNA
gctggaaatcaagcggaccg

SEQ ID NO: 8 - DNA, 20 bp, synthetic construct
  misc_feature 1..20: note = Synthetic: sgRNA #8 (eCHO8)
  source 1..20: mol_type = other DNA
tgatcatgtctcggggcaag

SEQ ID NO: 9 - DNA, 20 bp, synthetic construct
  misc_feature 1..20: note = Synthetic: sgRNA #9 (=#6) (eCHO9)
  source 1..20: mol_type = other DNA
gaacacggagggagcggcca

SEQ ID NO: 10 - DNA, 20 bp, synthetic construct
  misc_feature 1..20: note = Synthetic: sgRNA #10 (eCHO10)
  source 1..20: mol_type = other DNA
gtctcggggcaagcggaccg

SEQ ID NO: 11 - DNA, 20 bp, synthetic construct
  misc_feature 1..20: note = Synthetic: TIDE-F1
  source 1..20: mol_type = other DNA
taactcccgttgcggtgctg

SEQ ID NO: 12 - DNA, 20 bp, synthetic construct
  misc_feature 1..20: note = Synthetic: TIDE-R1
  source 1..20: mol_type = other DNA
ggaggacaggctgtaggtgc

SEQ ID NO: 13 - DNA, 20 bp, synthetic construct
  misc_feature 1..20: note = Synthetic: P1-Frw
  source 1..20: mol_type = other DNA
gtctgaaaatgagctcgggg

SEQ ID NO: 14 - DNA, 19 bp, synthetic construct
  misc_feature 1..19: note = Synthetic: VH-R for TIDE
  source 1..19: mol_type = other DNA
ggcttggtcttggcgttgt

SEQ ID NO: 15 - DNA, 20 bp, synthetic construct
  misc_feature 1..20: note = Synthetic: CM15_HC_F
  source 1..20: mol_type = other DNA
cctgatcctgctgttcctgg

SEQ ID NO: 16 - DNA, 20 bp, synthetic construct
  misc_feature 1..20: note = Synthetic: CM15_HC_R
  source 1..20: mol_type = other DNA
ccctaggtcatcacttgccg

SEQ ID NO: 17 - DNA, 29 bp, synthetic construct
  misc_feature 1..29: note = Synthetic: LMC98
  source 1..29: mol_type = other DNA
ggagttcaaagccagcttataccaacatg

SEQ ID NO: 18 - DNA, 25 bp, synthetic construct
  misc_feature 1..25: note = Synthetic: LMC101
  source 1..25: mol_type = other DNA
cctcttcttcagccacagtcacttc

SEQ ID NO: 19 - DNA, 27 bp, synthetic construct
  misc_feature 1..27: note = Synthetic: LMC102
  source 1..27: mol_type = other DNA
gctaaagttggtatggcagcctgcacc

SEQ ID NO: 20 - DNA, 20 bp, synthetic construct
  misc_feature 1..20: note = Synthetic: Ritux_FRT_F
  source 1..20: mol_type = other DNA
ccaagcccagagaggaacag

SEQ ID NO: 21 - DNA, 20 bp, synthetic construct
  misc_feature 1..20: note = Synthetic: Ritux_FRT_R
  source 1..20: mol_type = other DNA
gacaggtatccggtaagcgg

SEQ ID NO: 22 - DNA, 20 bp, synthetic construct
  misc_feature 1..20: note = Synthetic: Ritux_F5_F
  source 1..20: mol_type = other DNA
ttcattcagctccggttccc

SEQ ID NO: 23 - DNA, 20 bp, synthetic construct
  misc_feature 1..20: note = Synthetic: Ritux_F5_R
  source 1..20: mol_type = other DNA
gtgcccatctgttcccatca

SEQ ID NO: 24 - DNA, 20 bp, synthetic construct
  misc_feature 1..20: note = Synthetic: P1 Rev for TIDE
  source 1..20: mol_type = other DNA
tgaattcatcagcactcgcc

SEQ ID NO: 25 - DNA, 20 bp, synthetic construct
  misc_feature 1..20: note = Synthetic: VH-KI MN
  source 1..20: mol_type = other DNA
caccagctacaacatgcact

SEQ ID NO: 26 - DNA, 20 bp, synthetic construct
  misc_feature 1..20: note = Synthetic: Reverse MN
  source 1..20: mol_type = other DNA
cggagcaggagaacacgttg

SEQ ID NO: 27 - DNA, 20 bp, synthetic construct
  misc_feature 1..20: note = Synthetic: VL-KI MN
  source 1..20: mol_type = other DNA
ctggatctacgccacctcca

SEQ ID NO: 28 - DNA, 20 bp, synthetic construct
  misc_feature 1..20: note = Synthetic: Rev for VL insertion
  source 1..20: mol_type = other DNA
ggcggccgccagacatgata

SEQ ID NO: 29 - DNA, 20 bp, synthetic construct
  misc_feature 1..20: note = Synthetic: CL-D2-F1
  source 1..20: mol_type = other DNA
gttgaaggacttggtcacgg

SEQ ID NO: 30 - DNA, 20 bp, synthetic construct
  misc_feature 1..20: note = Synthetic: VH-R
  source 1..20: mol_type = other DNA
agtctttgaccaggcagccc

SEQ ID NO: 31 - DNA, 20 bp, synthetic construct
  misc_feature 1..20: note = Synthetic: SP-F
  source 1..20: mol_type = other DNA
gatgatctgcacctggaagt

SEQ ID NO: 32 - DNA, 20 bp, synthetic construct
  misc_feature 1..20: note = Synthetic: mCMV-R1
  source 1..20: mol_type = other DNA
catcgttctggtactgagtc

SEQ ID NO: 33 - DNA, 20 bp, synthetic construct
  misc_feature 1..20: note = Synthetic: sgRNA #11 (eCHO1)
  source 1..20: mol_type = other DNA
cgtgtctgccgcttccacca

SEQ ID NO: 34 - DNA, 20 bp, synthetic construct
  misc_feature 1..20: note = Synthetic: sgRNA #12 (eCHO2)
  source 1..20: mol_type = other DNA
gggggccagagggaacacgg

SEQ ID NO: 35 - DNA, 20 bp, synthetic construct
  misc_feature 1..20: note = Synthetic: sgRNA #13 (eCHO3)
  source 1..20: mol_type = other DNA
gaacacggaggggcccttgg

SEQ ID NO: 36 - DNA, 20 bp, synthetic construct
  misc_feature 1..20: note = Synthetic: sgRNA #14 (eCHO4)
  source 1..20: mol_type = other DNA
gaccaggaacagcaggatca

SEQ ID NO: 37 - DNA, 20 bp, synthetic construct
  misc_feature 1..20: note = Synthetic: sgRNA #15 (eCHO5)
  source 1..20: mol_type = other DNA
cctggtcgccgtggccaccc

SEQ ID NO: 38 - DNA, 20 bp, synthetic construct
  misc_feature 1..20: note = Synthetic: sgRNA #16 (eCHO6)
  source 1..20: mol_type = other DNA
cccgggtggccacggcgacc

SEQ ID NO: 39 - DNA, 20 bp, synthetic construct
  misc_feature 1..20: note = Synthetic: sgRNA #18
  source 1..20: mol_type = other DNA
agacagcacccgggtggcca

(19) United States
(12) Patent Application Publication
(10) Pub. No.: US 2025/0376661 A1
(43) Pub. Date: Dec. 11, 2025

(54) EDITABLE CELL LINES

(71) Applicant: LONZA SALES AG, Basel (CH)

(72) Inventors: Ariel GILERT, Basel (CH); Katarina RADOSEVIC, Basel (CH); Bracha SHRAIBMAN, Basel (CH); Frida Grynspan GOTLIEB, Basel (CH); Marc FEARY, Basel (CH); Bernadette SWEENEY, Basel (CH)

(21) Appl. No.: 18/876,556

(22) PCT Filed: Jun. 29, 2023
(86) PCT No.: PCT/US2023/069405

§ 371 Date: Dec. 18, 2024

Related U.S. Application Data
(60) Provisional application No. 63/367,419, filed on Jun. 30, 2022.

(51) Int. Cl. C12N 5/071; C07K 16/00; C12N 5/10; C12N 9/22; C12N 15/113

(52) U.S. Cl. CPC C12N 5/0682; C07K 16/00; C12N 5/10; C12N 9/226; C12N 15/113; C07K 2317/56; C12N 2510/02; C12N 2510/04

(57) ABSTRACT

The present disclosure provides editable cell lines, including the use of gene editing proteins to produce the cell lines. The editable cell lines are able to express antibody constant regions that can serve as a platform for the antibody variable regions to produce customized antibody.

### REFERENCE TO SEQUENCE LISTING SUBMITTED ELECTRONICALLY

The contents of the electronic sequence listing (0132-0194WO1_SL.xml; Size: 50,498 bytes; and Date of Creation Aug. 30, 2023) submitted herewith, is herein incorporated by reference in its entirety.

### FIELD OF THE INVENTION

The present disclosure provides editable cell lines, including the use of gene editing proteins to produce the cell lines. By preparing editable cell lines that contain the ability to be further modified to individually produce a desired antibody, the cost and time for antibody manufacturing process can be reduced.

### BACKGROUND OF THE INVENTION

As clinical adoption of advanced antibody therapies begins to gain traction, more attention is turning to the underlying manufacturing strategies that will allow these therapies to benefit patients worldwide. While antibody therapies hold great promise clinically, high manufacturing costs relative to reimbursement present a formidable roadblock to commercialization.

One of the challenges facing antibody therapies is the multi-staged and complex manufacturing process to produce the desired antibodies. Current manufacturing processes rely on introducing vectors to construct cell lines to express the gene of interest and to obtain the desired antibodies. This process requires vector contruction for each new antibody to be expressed followed by gene introduction and pool recovery. The process further requires clone selection to find high producing clones, which is time consuming with each clone requiring stability assessment and introduces the potential for deviations and failure.

What are needed to overcome these challenges are methods to shorten and simplify the manufacturing process. Editable cell lines are able to express antibody constant regions and can serve as a platform for the antibody variable regions and remove the need for vector construction and provide a reliable antibody manufacturing platform. The present invention fulfills these needs.

### SUMMARY OF THE INVENTION

In some embodiments, the disclosure provides a cell, comprising: a genomic nucleic acid sequence comprising, a first sequence encoding antibody heavy chain constant regions 1, 2 and 3, wherein the first sequence is not flanked by a sequence encoding an antibody heavy chain variable region, and a second sequence encoding antibody light chain constant region 1, wherein the second sequence is not flanked by a sequence encoding an antibody light chain variable region.

In further embodiments, provided is a method of producing an editable cell, comprising: providing a cell stably expressing a genomic nucleic acid sequence of an antibody that includes a variable heavy chain region sequence, constant heavy chain regions 1, 2 and 3 sequences, a variable light chain region sequence, and constant light chain region 1 sequence, excising the sequence encoding the variable heavy chain region with a gene editing protein, and excising the variable light chain region sequence with the gene editing protein.

Also provided herein is a method of making an antibody producing editable cell, comprising: providing a cell comprising a genomic nucleic acid sequence comprising, a first sequence encoding antibody heavy chain constant regions 1, 2 and 3, wherein the first sequence is not flanked by a sequence encoding an antibody heavy chain variable region, and a second sequence encoding antibody light chain constant region 1, wherein the second sequence is not flanked by a sequence encoding an antibody light chain variable region, introducing a sequence encoding an antibody heavy chain variable region to the cell, and introducing a sequence encoding an antibody light chain variable region to the cell.

### BRIEF DESCRIPTION OF THE DRAWINGS

FIG. 1. shows schematic representation of how an intermediate headless antibody can be modified with the introduction of antibody variable regions to create a desired customized antibody.

FIGS. 2A-2B show exemplary representations of producing an editable cell wherein the antibody heavy chain variable region and the antibody light chain variable region are excised from the genomic antibody sequence.

FIG. 3 shows an exemplary representation of genomic nucleic acid sequence wherein the sequence encoding the gene editing protein is introduced into the genomic nucleic acid sequence.

FIG. 4 shows knock out efficiency of various guide RNAs.

FIGS. 5A-5C show excision efficiency determined by PCR, FACS and NGS.

FIG. 6 shows editing efficiency of various guide RNAs.

FIGS. 7A-7C integration efficiency determined by PCR, FACS and NGS.

FIG. 8 shows cell viability assessment performed using Vi-Cell device.

FIG. 9 shows transfection efficiency determined by PCR.

FIGS. 10A-10C show integration efficiency determined by PCR, FACS and NGS.

FIGS. 11A-11B show light chain variable region knock-out efficiency of various gRNAs using TIDE analysis (11A) and PCR validation of excision of light chain variable region from existing antibody (11B).

FIGS. 12A-12C heavy chain variable region knock-out efficiency of various gRNAs using TIDE analysis (12A), PCR validation of excision of heavy chain variable region from delta VL antibody (12B), and PCR validation of heavy chain variable region from existing antibody (12C).

FIG. 13 shows schematic representation of generation of headless cell line from vector (BOB SSI).

FIGS. 14A-14B show cells' recovery following transfection with vector to generate headless cell line (14A) and FACS analysis showing the integration of the cassette into BOB cells using the SSI system (14B).

FIGS. 15A-15C show PCR validation of generation of headless cell line (15A), WB validation of generation of headless cell line (15B), and FACS analysis validating generation of headless cell line (15C).

FIG. 16 shows representation of DNA template used for heavy chain variable region-Puromycin integration.

FIGS. 17A-17B show cells' recovery following transfection of heavy chain variable region and Puromycin antibiotic selection (17A) and PCR validation showing the integration of heavy chain variable region (17B).

FIG. 18 shows representation of DNA template used for light chain variable region-Blasticidin integration.

FIGS. 19A-19B show cells' recovery following transfection of light chain variable region and Blasticidin antibiotic selection in delta VH cells (19A) and PCR validation showing the integration of light chain variable region in delta VH cells (19B).

FIGS. 20A-20D show successful transduction, selection, proliferation and cell banking of inducible-Cas9 BOB (SSI) cell line (20A), RT-PCR analysis showing Cas9 mRNA expression following induction with DOX (20B), ELISA analysis showing Cas9 protein expression following induction with DOX (20C), and WB showing Cas9 protein expression following induction with DOX (20D).

FIGS. 21A-21B show PCR validation showing successful generation of delta VL iBOB cell line (21A) and FACS analysis validating successful generation of delta VL iBOB cell line (21B).

FIG. 21C shows PCR validation of seamless integration of light chain variable region into delta VL cells to generate full antibody in iCas9 BOB cell line.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, cells, and/or nucleic acids of the invention can be used to achieve any of the methods as described herein.

As described throughout, the subject of this disclosure is an editable cell line that is in embodiments, stable and in further embodiments, high producing, capable of further modification to individually produce a desired, customized antibody. The editable cell line includes the use of gene editing proteins to further modify the genomic sequence encoding the headless antibody structure. The editable cell line may or may not express the headless antibody structure because the headless antibody structure is an intermediate. However, once the editable cell line is fully modified with antibody variable regions, the cell line can be expressed to produce fully customized antibody protein.

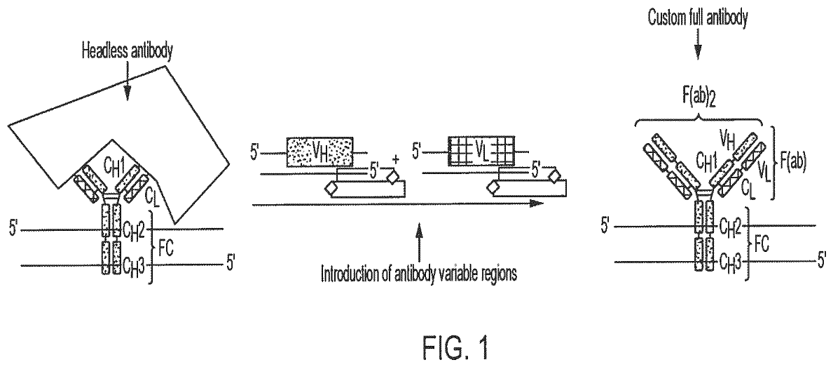

As used herein, “headless antibody” means an antibody protein without the antibody variable regions, but that includes constant heavy chain and light chain regions. The antibody variable regions include heavy chain and light chain variable regions that define the antigen binding site of the antibody protein. See, e.g., FIG. 1, showing a representation of an antibody that includes the heavy chain and light chain constant regions. The headless antibody structure is capable of modification and is only an intermediate, wherein antibody variable regions can be introduced to produce customized antibody proteins.

As used herein, “genomic nucleic acid” or “genomic sequence” means nucleic acids that are integrated into the genome of the cell. The term “genome” refers to the complete set of genetic information in the chromosomes of the cells.

As used herein, “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, gRNA, mRNA, tRNA, rRNA, snRNA, microRNA, miRNA, or MIRNA.

A “gene” as used herein refers to an assembly of nucleotides that encode a polypeptide and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

As used herein, “stable” means the cell line can maintain cell integrity with the use of commonly used storage methods and the cells are capable of maintaining antibody producing function over the course of multiple cell divisions. In embodiments, the cell lines described herein are stable cell lines. As used herein, “high producing” or “high expressing” means producing the molecule of interest in the amount of at least about 1 g/L. The amount that is considered high producing will depend on the molecule of interest being produced and may be about 2 g/L, about 3 g/L, about 4 g/L, about 5 g/L, about 6 g/L, about 7 g/L, about 8 g/L, about 9 g/L, about 10 g/L, about 15 g/L, about 20 g/L or higher. In embodiments, the cells provided herein are stable, high expressing cells.

FIG. 1 shows a schematic representation of how an intermediate headless antibody can be modified with the introduction of antibody variable regions to create a desired customized antibody. In FIG. 1, the overlapping antibody structure represents what is encoded and will be produced by expressing the DNA sequence. Accordingly, initially the sequence represents a headless antibody. Antibody variable regions are then introduced to the genome. Finally, the full structure of the customized antibody is expressed. Various methods for producing such headless antibody structures are described herein.

An editable cell line of this disclosure suitably does not require the use of DNA vectors to introduce the genetic sequence encoding the antibody for each antibody production. One commonly used method of producing antibody requires the use of recombinant DNA vectors, which are created, cloned, and introduced into the host cells for each antibody production cycle. However, this general method depends on the random/semi-random integration of the DNA vectors into the host cells.

An editable cell line can reduce the time and cost of the commonly used antibody production method by removing the need to produce DNA vectors, introducing the vectors to the host cells, and selecting the host cells with DNA vectors present to produce the antibodies. In the disclosed editable cell lines, the sequence encoding the headless antibody is integrated into the genomic sequence of the host cell. Therefore, once the editable cells are modified to produce the full antibody, said cells can be selected and cloned to produce antibodies.

An editable cell line can be produced by using an existing high antibody producing cell line, wherein the sequence encoding antibody variable regions are removed from the genomic sequence. The resulting cell line still contains the sequence encoding the rest of the antibody structure, which is the headless antibody.

In some embodiments, provided herein is a method of producing an editable cell, wherein the sequence encoding the variable heavy chain region and the sequence encoding the variable light chain region are excised from the genomic sequence encoding the full antibody of a suitably stable, and in embodiments, high expressing cell.

In some embodiments, provided herein is a method of producing an editable cell, comprising: providing a cell stably expressing a genomic nucleic acid sequence of an antibody that includes a variable heavy chain region sequence, constant heavy chain regions 1, 2, and 3 sequences, a variable light chain region sequence, and constant light chain region 1 sequence, excising the sequence encoding the variable heavy chain region with a gene editing protein, and excising the variable light chain region sequence with the gene editing protein. In some embodiments, the sequence encoding the variable heavy chain region is excised with a gene editing protein before the sequence encoding the variable light chain region is excised. In some embodiments, the sequence encoding the variable light chain region is excised with a gene editing protein before the sequence encoding the variable heavy chain region is excised. In some embodiments, the sequence encoding the variable heavy chain region and the sequence encoding the variable light chain region are excised simultaneously.

An editable cell line can also be produced by using a targeting vector encoding a headless antibody to integrate the sequence into the genomic sequence of an existing high antibody producing cell. The resulting cell can be selected and cloned to produce a cell line capable of expressing the headless antibody and modified to produce full antibody.

In some embodiments, provided herein is a method of producing an editable cell, wherein the sequence encoding the headless antibody is integrated into the genomic sequence of a suitably stable, and in embodiments, high expressing cell. In some embodiments, the sequence encoding the headless antibody integrated into the genomic sequence of the cell through site-specific integration of a vector containing the sequence.

In some embodiments, provided herein is a method of producing an editable cell, further comprising: introducing a sequence encoding the gene editing protein to the genomic nucleic acid sequence prior to excising the sequence encoding the variable heavy chain region and the variable light chain region; and expressing the gene editing protein prior to excising the variable heavy chain region and the variable light chain region. In some embodiments, provided herein is a method of producing an editable cell, further comprising: introducing a ribonucleoprotein (RNP) of the suitable gene editing protein prior to the excising the sequence encoding the variable heavy chain region and the variable light chain region. In some embodiments, provided herein is a method of producing an editable cell, further comprising: introducing a plasmid containing the sequence encoding the gene editing protein to the cell prior to excising the sequence encoding the variable heavy chain region and the variable light chain region; and expressing the gene editing protein sequence in the plasmid prior to excising the variable heavy chain region and the variable light chain region.

As used herein, the terms “engineered nuclease”, “engineered gene editing protein”, or “gene editing protein” refer to a nuclease that has been separated, modified, mutated, and/or altered from its natural state as a nuclease. A “nuclease” refers to an enzyme that is able to cut a DNA and/or RNA molecule. By engineering the nuclease, the specific location of the cut can be designed and tailored to the desired cell type and/or gene of interest.

Exemplary engineered nucleases that can be inserted into the cell (either produced from integrated, genomic nucleic acids, viral or other non-genomic nucleic acids, or as RNP) include, for example, a meganuclease, a methyltransferase a zinc finger nuclease, a transcription activator-like effector-based nuclease (TALENS), a FokI nuclease, and a CRISPR-associated (Cas) nuclease. In general, engineered nucleases use a DNA-binding protein which has both a desired catalytic activity and the ability to bind the desired target sequence through a protein-nucleic-acid interaction in a manner similar to restriction enzymes. Examples include meganucleases which are naturally occurring or engineered rare sequence cutting enzymes, zinc finger nucleases (ZFNs) or transcription activator-like nucleases (TALENs) which contain the FokI catalytic nuclease subunit linked to a modified DNA binding domain and can cut one predetermined sequence each. In ZFNs the binding domain is comprised of chains of amino-acids folding into customized zinc finger domains. In TALENs, similarly, 34 amino acid repeats originating from transcription factors fold into a huge DNA-binding domain. In the event of gene targeting, these enzymes can cleave genomic DNA to form a double strand break (DSB) or create a nick which can be repaired by one of two repair pathways, non-homologous end joining (NHEJ) or homologous recombination (HR). The NHEJ pathway can potentially result in specific mutations, deletions, insertions or replacement events. The HR pathway results in replacement of the targeted sequence by a supplied donor sequence. Exemplary FokI and methyltransferase-based systems are described in U.S. Pat. No. 10,220,052, the disclosure of which is incorporated by reference herein in its entirety.

The Clustered Regularly Interspaced Short Palindromic Repeats (CRISPR) and associated proteins (CRISPR-associated nucleases, or Cas proteins), which comprise the CRISPR-Cas system, were first identified in selected bacterial species and form part of a prokaryotic adaptive immune system. See Sorek, et al., “CRISPR—a widespread system that provides acquired resistance against phages in bacteria and archaea,” Nat. Rev. Microbiol. 6 (3) 181-6 (2008), which is incorporated by reference herein in its entirety. CRISPR-Cas systems have been classified into three main types: Type I, Type II, and Type III. The main defining features of the separate Types are the various cas genes, and the respective proteins they encode, that are employed. The cas1 and cas2 genes appear to be universal across the three main Types, whereas cas3, cas9, and cas 10 are thought to be specific to the Type I, Type II, and Type III systems, respectively. See, e.g., Barrangou, R. and Marraffini, L. A., “CRISPR-Cas systems: prokaryotes upgrade to adaptive immunity,” Mol. Cell. 54 (2): 234-44 (2014), which is incorporated by reference herein in its entirety.

In general, the CRISPR-Cas system functions by capturing short regions of invading viral or plasmid DNA and integrating the captured DNA into the host genome to form so-called CRISPR arrays that are interspaced by repeated sequences within the CRISPR locus. This acquisition of DNA into CRISPR arrays is followed by transcription and RNA processing.

Depending on the bacterial species, CRISPR RNA processing proceeds differently. For example, in the Type II system, originally described in the bacterium Streptococcus pyogenes, the transcribed RNA is paired with a transactivating RNA (tracrRNA) before being cleaved by RNase III to form an individual CRISPR-RNA (crRNA). The crRNA is further processed after binding by the Cas9 nuclease to produce the mature crRNA. The crRNA/Cas9 complex subsequently binds to DNA containing sequences complimentary to the captured regions (termed protospacers). The Cas9 protein then cleaves both strands of DNA in a site-specific manner, forming a double-strand break (DSB). This provides a DNA-based memory, resulting in rapid degradation of viral or plasmid DNA upon repeat exposure and/or infection.

Since its original discovery, multiple groups have done extensive research around potential applications of the CRISPR system in genetic engineering, including gene editing (Jinek et al., “A programmable dual-RNA-guided DNA endonuclease in adaptive bacterial immunity,” Science 337 (6096): 816-21 (2012); Cong et al., “Multiplex genome engineering using CRISPR/Cas systems,” Science 339 (6121): 819-23 (2013); and Mali et al., “RNA-guided human genome engineering via Cas9,” Science 339 (6121): 823-26; each of which is incorporated by reference herein in its entirety). One major development was utilization of a chimeric RNA to target the Cas9 protein, designed around individual units from the CRISPR array fused to the tracrRNA. This creates a single RNA species, called a small guide RNA (gRNA) where modification of the sequence in the protospacer region can target the Cas9 protein site-specifically. Considerable work has been done to understand the nature of the base-pairing interaction between the chimeric RNA and the target site, and its tolerance to mismatches, which is highly relevant in order to predict and assess off-target effects (see, e.g., Fu et al., “Improving CRISPR-Cas nucleases using truncated guide RNAs,” Nature Biotechnology 32 (3): 279-84 (2014), and supporting material, which is incorporated by reference herein in its entirety).

The CRISPR-Cas9 gene editing system has been used successfully in a wide range of organisms and cell lines, both in order to induce double-strand break formation using the wild type Cas9 protein or to nick a single DNA strand using a mutant protein termed Cas9n/Cas9 D10A (see, e.g., Mali et al., (2013) and Sander and Joung, “CRISPR-Cas systems for editing, regulating and targeting genomes,” Nature Biotechnology 32 (4): 347-55 (2014), each of which is incorporated by reference herein in its entirety). While double-strand break (DSB) formation results in creation of small insertions and deletions (indels) that can disrupt gene function, Cas9 wild-type as well as Cas9n/Cas9 D10A nickase can avoid indel creation (the result of repair through non-homologous end-joining) while stimulating the endogenous homologous recombination machinery. Thus, these systems can be used to insert regions of DNA into the genome with high-fidelity.

In some embodiments, provided herein is a method of producing an editable cell, wherein the gene editing protein utilized in the methods to excise variable regions, is a CRISPR-associated gene editing protein. In suitable embodiments, the CRISPR-associated (Cas) nuclease is a Cas9 nuclease, or can be other Cas nucleases such as Cas12, Cas1212, Cas13, Cas14, MAD7 (Cas12a), etc. In some embodiments, the Cas9 nuclease is a Cas9 nuclease that has reduced immunogenicity, such as disclosed in U.S. Published Patent Application No. 2018-0319850, the disclosure of which is incorporated by reference herein in its entirety. In some embodiments, the gene editing protein is a zinc finger nuclease. In some embodiments, the gene editing protein is a TALENS. In some embodiments, the gene editing protein is a FokI nuclease.

In addition to a Cas9 nuclease, Cas12, Cas13, Cas14, and MAD7 (Cas12a) nucleases can also be utilized in the methods described herein. Cas12 creates staggered cuts in dsDNA (5 nucleotide 5′ overhang dsDNA break). Cas12 processes its own guide RNAs, leading to increased multiplexing ability. Cas13t targets RNA, not DNA. Once it is activated by a ssRNA sequence bearing complementarity to its crRNA spacer, it unleashes a nonspecific RNase activity and destroys all nearby RNA regardless of their sequence. See, e.g., Yan et al., “CRISPR-Cas12 and Cas13: the lesser known siblings of CRISPR Cas9,” Cell Biology and Toxicology pages 1˜4 (Aug. 29, 2019), the disclosure of which is incorporated by reference herein in its entirety. In some embodiments, the Cas1212 nuclease can also utilized in the methods described herein, such as disclosed in U.S. Pat. No. 10,808,245, the disclosure of which is incorporated by reference herein in its entirety.

In further embodiments, provided herein is a method of producing an editable cell, wherein the gene editing protein is used to excise the sequences from the genomic sequence of the cell. In some embodiments, excising the sequence encoding the variable heavy chain region with the gene editing protein occurs at a first guide RNA target sequence and a second guide RNA target sequence; and excising the variable light chain region sequence with the gene editing protein occurs at a third guide RNA target sequence and a fourth guide RNA target sequence. In some embodiments, the method further comprises introducing a first guide RNA and a second guide RNA. In some embodiments, the method further comprises introducing a third guide RNA and a fourth guide RNA. In some embodiments, the method further comprises introducing guide RNAs through plasmid that expresses the guide RNA transiently.

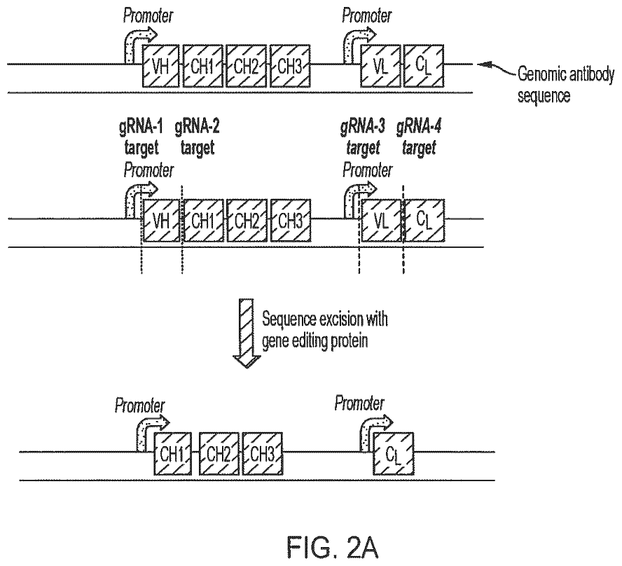

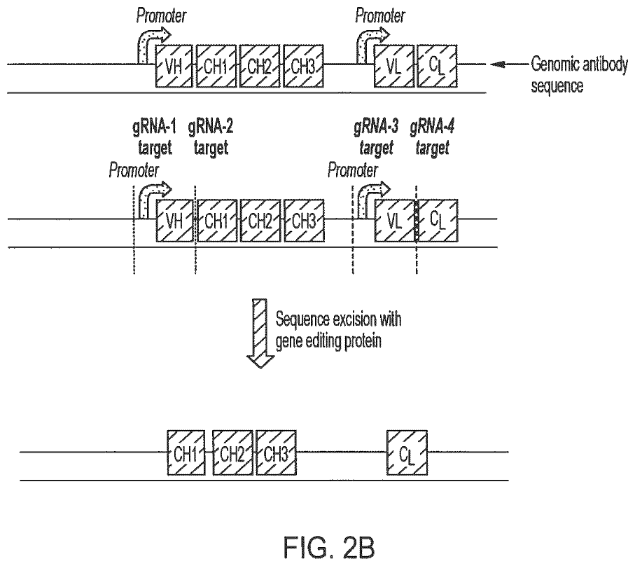

FIGS. 2A-2B show representations of a method of producing an editable cell by excising the sequences encoding the antibody variable regions from the genomic antibody sequence. VH represents the sequence encoding variable heavy chain region and VL represents the sequence encoding variable light chain region of the antibody. CH1, CH2, and CH3 represent the sequences encoding constant heavy regions 1, 2, and 3, respectively, of the antibody. CL represents the sequence encoding constant light region of the antibody. CMV represents an exemplary promoter sequence and the arrow indicates the direction of gene expression. As indicated in FIG. 2A, the sequences encoding the antibody variable regions are excised from the genomic sequence through the use of a gene editing protein, suitably targeting the first gRNA and second gRNA target sequences to excise VH and targeting the third gRNA and fourth gRNA target sequences to excise VL. In FIG. 2B, the antibody is not expressed, as the promoter regions have been excised. In other embodiments, guide RNA sequences are not required, depending on the selection of the corresponding gene editing proteins.

In some embodiments, provided herein is a method of producing an editable cell, wherein the sequence encoding the gene editing protein (genomically integrated) is operably connected to an inducible promoter. By placing the gene editing protein under the control of an inducible promoter, the nuclease can be kept dormant or silent prior to its desired use as a gene editing tool. In some embodiments, the inducible promoter is a TET-on system.

As used herein, a “promoter,” “promoter sequence,” or “promoter region,” which refers to a DNA regulatory region/sequence capable of binding RNA polymerase and initiating transcription of a downstream coding or non-coding gene sequence. In other words, the promoter and the gene are in operable combination or operably linked. As referred to herein, the terms “in operable combination”, “in operable order”, “operably connected”, and “operably linked” refer to the linkage of nucleic acid sequences in such a manner that a promoter capable of directing the transcription of a given gene and/or the synthesis of a desired protein molecule is produced. The term also refers to the linkage of amino acid sequences in such a manner so that a protein is produced.

In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

Various promoters may be used to drive the gene expression. In some embodiments, the promoter is an “inducible promoter”, i.e., the promoter is not constitutively expressing any of the gene products as described herein and is activated in response to specific stimuli that can be turned on or off, depending on the desired control of the gene that is under control of the promoter. In other embodiments as described herein, the promoter is a constitutive promoter, which initiates mRNA synthesis independent of the influence of an external regulation.

Suitably, the promoters used to control the engineered nucleases are derepressible promoters. As used herein, a “derepressible promoter” refers to a structure that includes a functional promoter and additional elements or sequences capable of binding to a repressor element to cause repression of the functional promoter. “Repression” refers to the decrease or inhibition of the initiation of transcription of a downstream coding or non-coding gene sequence by a promoter. A “repressor element” refers to a protein or polypeptide that is capable of binding to a promoter (or near a promoter) so as to decrease or inhibit the activity of the promoter. A repressor element can interact with a substrate or binding partner of the repressor element, such that the repressor element undergoes a conformation change. This conformation change in the repressor element takes away the ability of the repressor element to decrease or inhibit the promoter, resulting in the “derepression” of the promoter, thereby allowing the promoter to proceed with the initiation of transcription. A “functional promoter” refers to a promoter, that absent the action of the repressor element, would be capable of initiation transcription. Various functional promoters that can be used in the practice of the present invention are known in the art, and include for example, PCMV, PH1, P19, P5, P40 and promoters of Adenovirus helper genes (e.g., E1A, EIB, E2A, E4Orf6, and VA).

Examples of various controllable promoters, including inducible promoters and derepressible promotors are described herein, as are methods of inducing expression of the Cas9 nuclease via the introduction of a molecule that induces expression, or that derepresses a derepressible promoter.

Exemplary repressor elements and their corresponding binding partners that can be used as derepressible promoters are known in the art, and include systems such as the cumate gene-switch system (CuO operator, CymR repressor and cumate binding partner) (see, e.g., Mullick et al., “The cumate gene-switch: a system for regulated expression in mammalian cells,” BMC Biotechnology 6:43 (1-18) (2006), the disclosure of which is incorporated by reference herein in its entirety, including the disclosure of the derepressible promoter system described therein) and the TetO/TetR system described herein (see, e.g., Yao et al., “Tetracycline Repressor, tetR, rather than the tetR-Mammalian Cell Transcription Factor Fusion Derivatives, Regulates Inducible Gene Expression in Mammalian Cells,” Human Gene Therapy 9:1939-1950 (1998), the disclosure of which is incorporated by reference herein in its entirety). In exemplary embodiments, the derepressible promoters comprise a functional promoter and either one two tetracycline operator sequences (TetO or TetO₂). In such embodiments, the nucleic acid introduced into the T-cells further includes a tetracycline repressor protein to control the TetO derepressible system (a TET-on system).

As described herein, the methods can further include inducing expression of the CRISPR-associated nuclease by activating the inducible promoter. In the case of an inducible promoter, such as a 4HT inducible promoter, a rapamycin inducible promoter, a hormone response element, or a glutamate inducible promoter, the promoter is induced by the addition of, for example, 4-hydroxytamoxifen, rapamycin, a hormone, or glutamate, respectively. In the case of a derepressible promoter, such as the TetO sequence described herein coupled to a CMV promoter, the addition of doxycycline removes the repression, and allows the gene (engineered nuclease) to be expressed via the CMV promoter. Suitably, the nucleic acid molecule that encodes the Cas9 also encodes a TetR repressor element, suitably under the control of another promoter system, such as a constitutive promoter like the hPGK promoter.

In further embodiments, provided herein is a method of making an antibody producing cell, comprising: providing a cell as described herein, introducing a sequence encoding an antibody heavy chain variable region and a fifth guide RNA target sequence to the genomic nucleic acid sequence, and introducing a sequence encoding an antibody light chain variable region and a sixth guide RNA target sequence to the genomic nucleic acid sequence. In some embodiments, the sequence encoding an antibody heavy chain variable region and the fifth guide RNA target sequence is operably connected to the first sequence encoding antibody heavy chain constant regions 1, 2, and 3. In some embodiments, the sequence encoding an antibody light chain variable region and the sixth guide RNA target sequence are operably connected to the second sequence encoding antibody light chain constant region 1.

In some embodiments, provided herein is a method of making an antibody producing cell, further comprising introducing the sequence encoding an antibody heavy chain variable region and the fifth guide RNA target sequence to the upstream of the first sequence encoding antibody heavy chain constant regions 1, 2, and 3. In some embodiments, provided herein is a method of making an antibody producing cell, further comprising introducing the sequence encoding an antibody light chain variable region and the sixth guide RNA target sequence to the upstream of the second sequence encoding antibody light chain constant region 1. In some embodiments, the method further comprises introducing a promoter sequence with the sequence encoding an antibody heavy chain variable region. In some embodiments, the method further comprises introducing a promoter sequence with the sequence encoding an antibody light chain variable region. In some embodiments, the promoter sequence is operably connected to the sequence encoding an antibody heavy chain variable region. In some embodiments, the sequence encoding an antibody heavy chain variable region is operably connected to the first sequence encoding antibody heavy chain constant regions 1, 2, and 3. In some embodiments, the promoter sequence is operably connected to the sequence encoding an antibody light chain variable region. In some embodiments, the sequence encoding an antibody light chain variable region is operably connected to the second sequence encoding antibody light chain constant region 1.

In some embodiments, the method further comprises introducing a sequence encoding a selectable marker.

As used herein, the term “selectable marker” or “selectable marker gene” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin (blast), and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2.alpha.; detectable by Mab-FITC); Methylguanine-DNA methyltransferase (MGMT; selectable by carmustine); rhamnose; and Cytidine deaminase (CD; selectable by Ara-C).

In some embodiments, provided herein is a method of producing an editable cell, wherein the gene editing protein is a CRISPR-associated gene editing protein. In suitable embodiments, the CRISPR-associated (Cas) nuclease is a Cas9 nuclease, or can be other Cas nucleases such as Cas12, Cas1212, Cas13, Cas14, MAD7 (Cas12a), etc. In some embodiments, the Cas9 nuclease is a Cas9 nuclease that has reduced immunogenicity, In some embodiments, the gene editing protein is a zinc finger nuclease. In some embodiments, the gene editing protein is a TALENS. In some embodiments, the gene editing protein is a FokI nuclease.

In some embodiments, provided herein is a method of producing an editable cell, wherein the sequence encoding the gene editing protein is operably connected to an inducible promoter. By placing the gene editing protein under the control of an inducible promoter, the nuclease can be kept dormant or silent prior to its desired use as a gene editing tool. In some embodiments, the inducible promoter is a TET-on system, as described herein.

In some embodiments, provided herein is a method of producing an editable cell, further comprising introducing a first promoter operably connected to the sequence encoding constant heavy chain region 1 and introducing a second promoter operably connect to the sequence encoding constant light chain region 1.

As described herein, in embodiments, the editable cell is a eukaryotic cell. In some embodiments, the cell is a mammalian cell, a yeast cell, an insect cell, an algae cell, or a plant cell. In suitable embodiments, the cell is a Chinese hamster ovary (CHO) cell. Examples of CHO cells include, but are not limited to, CHO-K1, CHOK1SV, Potelligent CHOK1SV (FUT8-KO), CHO GS-KO, Xceed (CHOK1SV GS-KO), CHO-S, CHO DG44, CHO DXB11, CHOZN, HD7876 BOB cells which are GFP positive CHO cells or a CHO-derived cell. In some embodiments, the cell is a Chinese Hamster Ovary (CHO) cell.

In suitable embodiments, the cell is a human embryonic kidney (HEK) cell. In some embodiments, the cell is selected from the group consisting of HeLa, HEK293, H9, HepG2, MCF7, Jurkat, NIH3T3, PC12, PER.C6, BHK, VERO, SP2/0, NS0, YB2/0, EB66, C127, L cell, COS, e.g., COS1 and COS7, QC1-3, CHO-K1, CHOK1SV, Potelligent CHOK1SV (FUT8-KO), CHO GS-KO, Exceed (CHOK1SV GS-KO), CHO-S, CHO DG44, CHO DXB11, and CHOZN.

In further embodiments, provided herein is a cell, comprising a genomic nucleic acid sequence comprising, a first sequence encoding antibody heavy chain constant regions 1, 2, and 3, wherein the first sequence is not flanked by a sequence encoding an antibody heavy chain variable region, and a second sequence encoding antibody light chain constant region 1, wherein the second sequence is not flanked by a sequence encoding an antibody light chain variable region. See for Example, FIG. 2A and FIG. 2B, illustrating an embodiment where the heavy chain and light chain regions are on the same genomic sequence. In embodiments, the first sequence and the second sequence can be on the same section of genomic DNA, including the same DNA strand. However, in other embodiments, the first and second sequences can be on different DNA strands, including being on separate chromosomes. In addition, the first sequence and the second sequence can be in any order.

In some embodiments, the cell further comprises a first promoter operably connected to the first sequence and a second promoter operably connected to the second sequence. In some embodiments, the cell further comprises a single promoter operably connected to either the first sequence or the second sequence and wherein the first and the second sequences are operably connected to each other. In some embodiments, the first and the second sequences do not have a promoter connected to the sequence.

In some embodiments, the cell further comprises a sequence encoding a gene editing protein. In some embodiments, the gene editing protein is a CRISPR-associated gene editing protein. In suitable embodiments, the CRISPR-associated (Cas) nuclease is a Cas9 nuclease, or can be other Cas nucleases such as Cas12, Cas1212, Cas13, Cas14, MAD7 (Cas12a), etc. In some embodiments, the Cas9 nuclease is a Cas9 nuclease that has reduced immunogenicity. In some embodiments, the gene editing protein is a zinc finger nuclease. In some embodiments, the gene editing protein is a TALENS. In some embodiments, the gene editing protein is a FokI nuclease. As described herein, the gene editing protein can be included as a part of the genomic sequence, or can be a separately provided sequence (e.g., via a vector) or as an RNP.

In some embodiments, the sequence encoding the gene editing protein is operably connected to an inducible promoter. By placing the gene editing protein under the control of an inducible promoter, the nuclease can be kept dormant or silent prior to its desired use as a gene editing tool. In some embodiments, the inducible promoter is a TET-on system, as described herein.

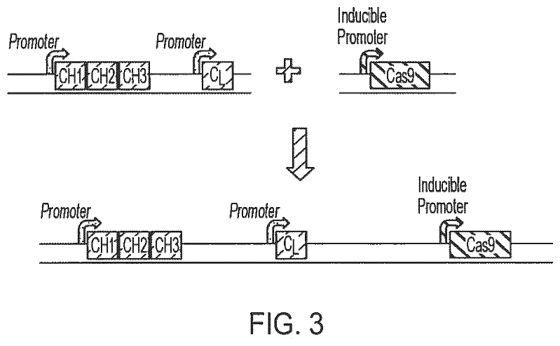

FIG. 3 shows an exemplary schematic representation of a genomic sequence of the editable cell comprising the first and the second sequences as well as the sequence encoding the gene editing protein (e.g., Cas9 as shown). The gene editing protein is suitably operably connected to an inducible promoter so the gene editing protein can be kept dormant prior to its desired use as a gene editing tool.

As described herein, the cell is suitably a eukaryotic cell. In some embodiments, the cell is a mammalian cell, a yeast cell, an insect cell, an algae cell, or a plant cell. In suitable embodiments, the cell is a Chinese hamster ovary (CHO) cell. Examples of CHO cells include, but are not limited to, CHO-K1, CHOK1SV, Potelligent CHOK1SV (FUT8-KO), CHO GS-KO, Xceed (CHOK1SV GS-KO), CHO-S, CHO DG44, CHO DXB11, CHOZN, or a CHO-derived cell. In some embodiments, the cell is a Chinese Hamster Ovary (CHO) cell. In suitable embodiments, the cell is a human embryonic kidney (HEK) cell. In some embodiments, the cell is selected from the group consisting of HeLa, HEK293, H9, HepG2, MCF7, Jurkat, NIH3T3, PC12, PER.C6, BHK, VERO, SP2/0, NS0, YB2/0, EB66, C127, L cell, COS, e.g., COS1 and COS7, QC1-3, CHO-K1, CHOK1SV, Potelligent CHOK1SV (FUT8-KO), CHO GS-KO, Exceed (CHOK1SV GS-KO), CHO-S, CHO DG44, CHO DXB11, and CHOZN.

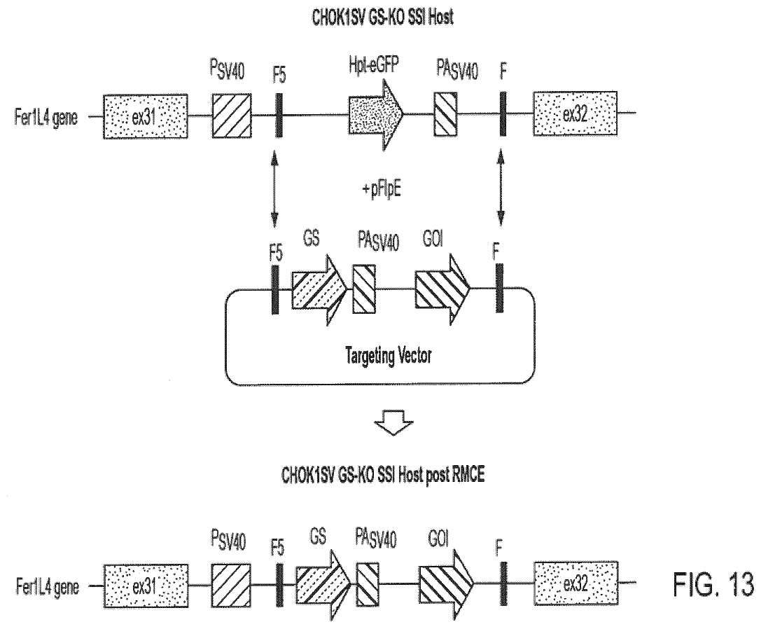

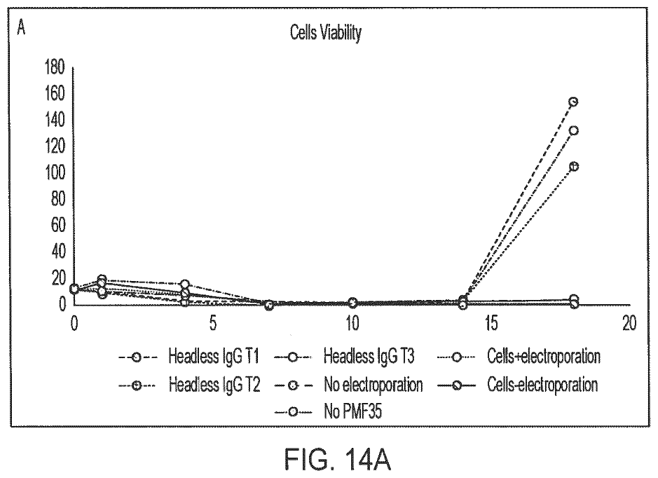

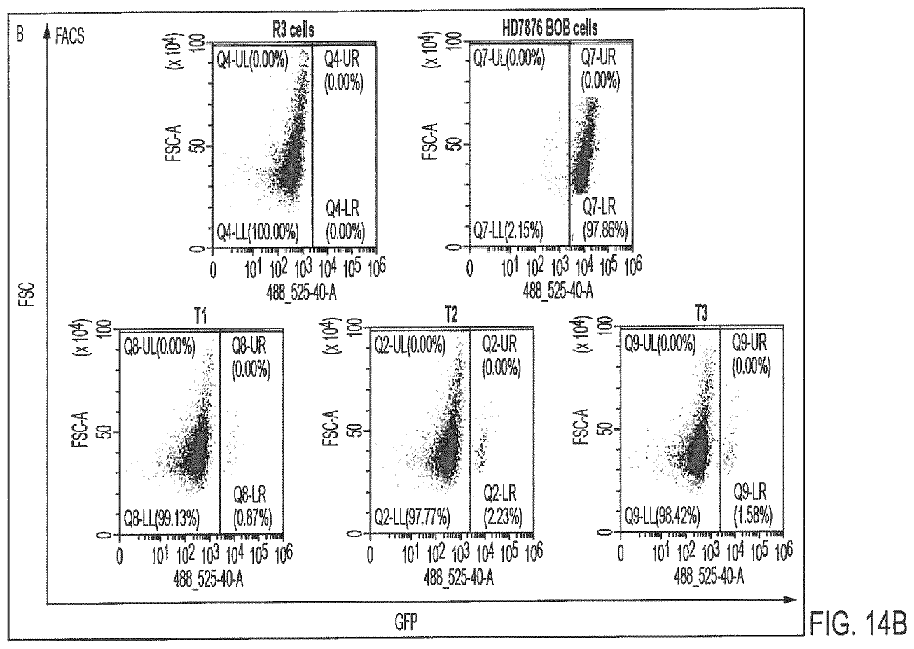

FIG. 13 shows schematic representation of generation of headless cell line by site-specific integration of the vector encoding the headless antibody into the genomic sequence of the host cell. FIG. 14A shows cells' recovery following transfection with vector to generate headless antibody cell line. As shown, the headless antibody sequences are successfully integrated into the genomic sequence as cell lines treated with the vectors show an increase in cell viability compared to the controls. FIG. 14B shows FACS analysis, validating cassette integration.

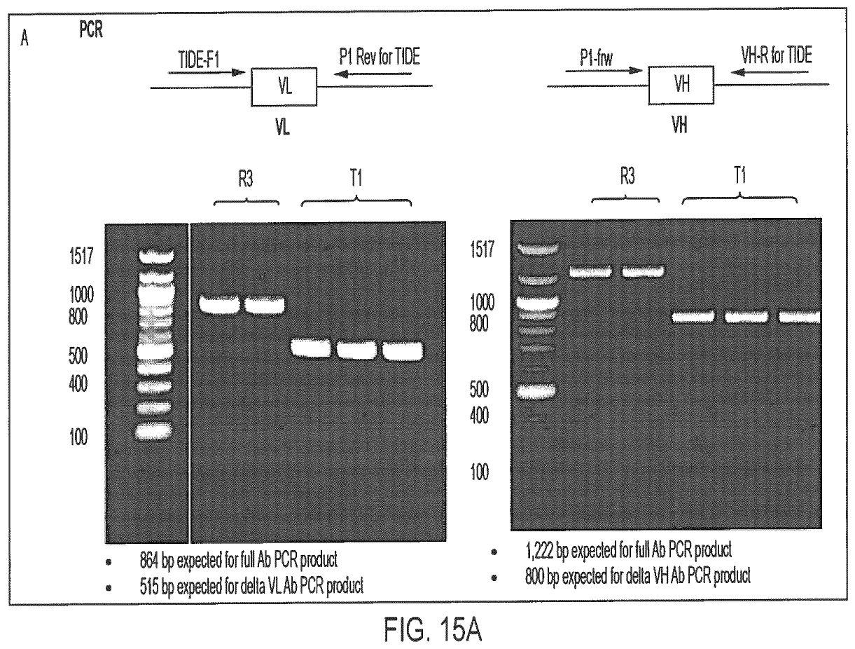

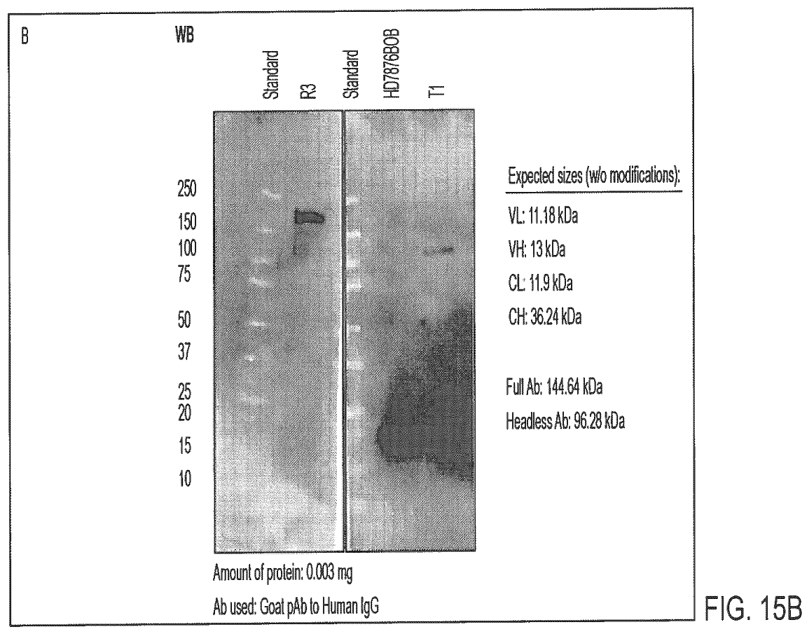

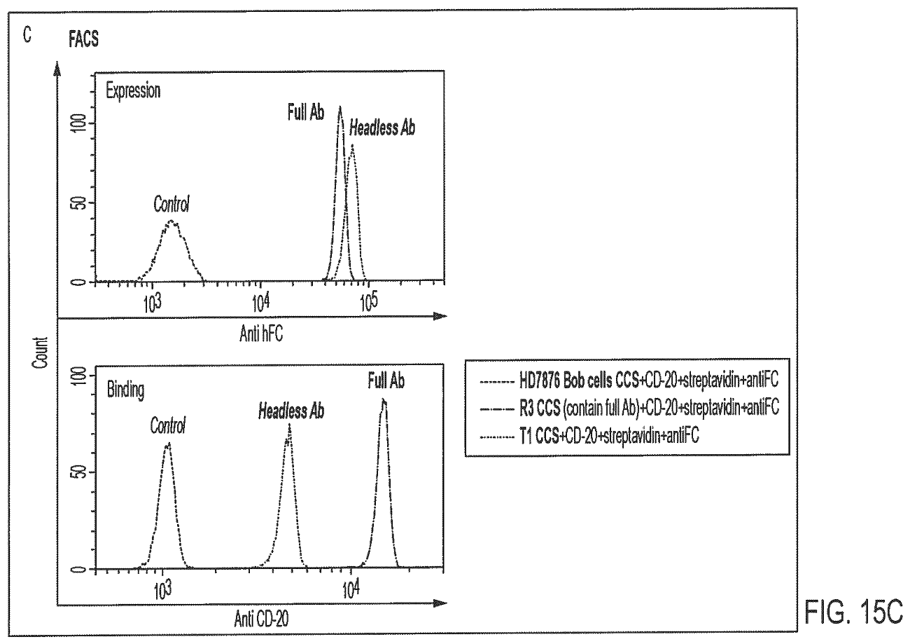

FIGS. 15A-15C show further validation of successful generation of a headless antibody cell line from vector. FIG. 15A shows validation by PCR as indicated by bands at 515 bp for delta VL compared to bands at 864 bp for full ab product and bands at 800 bp for delta VH compared to bands at 1,222 bp for full antibody product. FIG. 15B shows validation by Western Blot as indicated by the band for full antibody in the left column compared to the band for headless antibody in the right column. FIG. 15C shows validation by FACS analysis wherein the headless antibody is expressed but does not bind to CD20 antigen compared to the full antibody.

In some embodiments, the cell further comprises a sequence encoding a selectable marker.

In some embodiments, provided herein is a method of making an antibody producing cell, further comprising introducing a promoter operably connected to the sequence encoding the antibody heavy chain region and a promoter operably connected to the sequence encoding the antibody light chain region. In some embodiments, the method further comprises introducing a promoter operably connected to the sequence encoding antibody.

In some embodiments, provided herein is a method of making an antibody producing cell, further comprising introducing a first sequence encoding a first selectable marker and a second sequence encoding a second selectable marker. In some embodiments, the method further comprises selecting a cell expressing the antibody using the first selectable marker. In some embodiments, the method further comprises selecting a cell expressing the antibody using the second selectable marker. In some embodiments, the method further comprises selecting a cell expressing the antibody using the first and the second selectable markers. In some embodiments, the method further comprises expressing the antibody in the cell.

Methods for expanding the cells produced using the methods described herein are known in the art, as are methods for recovering the antibodies produced thereby. Such methods, include for example, various column filtration methods, washing steps, as well as bead-based magnetic separation methods, etc.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

### EXAMPLES

### Example 1: Selecting sgRNAs for Excision of Light Chain Variable Region from Full IgG

### Method

SSI Cells Sub-Culturing and sgRNA Preparation

Site Specific Integration (SSI) cells (see, e.g., WO 2020/072480) expressing a full IgG antibody were sub-cultured at a viable concentration of 0.3×10⁶ cells/mL into an appropriately sized Erlenmeyer shake-flask (with CD-CHO medium supplemented with 6 mM L-glutamine). These cells were in culture for no less than 4 days and no more than 4 weeks and have viability greater than 90% prior to transfection.

sgRNAs (TrueGuide Synthetic sgRNA 3 nmol (Invitrogen. Cat No. A35514)) were centrifuged briefly to collect contents and re-suspended to 100 μM in nuclease-free Tris-EDTA (TE) buffer to generate a stock solution. A 10 μM working solution was generated from this stock. All sgRNA solutions were stored at −20° C. Following 7 sgRNA sequences were prepared.

| Guide name | Sequence (5′ to 3′) |
|---|---|
| sgRNA #1 (eCHO1) | GCCCCGAGACATGATCACGG |
| | (SEQ ID NO: 1) |
| sgRNA #2 (eCHO2) | CCGCCTCCGTGATCATGTCT |
| | (SEQ ID NO: 2) |
| sgRNA #3 (eCHO3) | GCCTCCGTGATCATGTCTCG |
| | (SEQ ID NO: 3) |
| sgRNA #4 (eCHO4) | CTTCGGCGGAGGCACCAAGC |
| | (SEQ ID NO: 4) |
| sgRNA #5 (eCHO5) | GCACCAAGCTGGAAATCAAG |
| | (SEQ ID NO: 5) |
| sgRNA #6 (eCHO6) | GAACACGGAGGGAGCGGCCA |
| | (SEQ ID NO: 6) |
| sgRNA #7 (eCHO7) | GCTGGAAATCAAGCGGACCG |
| | (SEQ ID NO: 7) |

### Nucleofection

The general nucleofection conditions, per individual transfection, were as follows:

| | |
|---|---|
| Amount of Cell | 2.5 × 10\hat{ }5 cells |
| TrueCut Cas9 Protein V2 | 4 ug (0.8 ul) |
| (Invitrogen. Cat No. A36499) | |
| sgRNA 10 uM | 0.8 ul (Cas9:sgRNA ratio is 1:0.3) |
| Nucleofection solution | SF solution supplemented |
| | with supplement 1 (82% v/v SF |
| | solution, 18% v/v supplement 1); |
| | 18.4 ul per transfection. |

Cas9 RNP complex mixtures for each sgRNA sequences above were prepared in 7 Eppendorf tubes by mixing 3.2 μl of Cas9 protein with 3.2 μl of 10 μM sgRNA. 3 control mixtures were also prepared with 1 Eppendorf tube containing 3.2 μl of Cas9 protein and 3.2 μl of TE buffer and 2 Eppendorf tubes each containing 6.4 μl of TE buffer. The mixtures were incubated at room temperature for 30-60 minutes.

SSI cells expressing full IgG antibody were prepared for nucleofection by transferring 10⁷ cells to a new tube and centrifuged at 300 g for 5 min to remove as much media as possible. The cells were then re-suspended in 736 μl of Nucleofection solution (656 μl of SF solution with 144 μl of supplement 1). 73.6 μl of cells were added to each of the 10 Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium supplemented with 6 mM L-glutamine warmed to a 36.5° C. 20 μl of the final mixtures were transferred to the 96-well plate with 100 μl of a CD-CHO medium supplemented with 6 mM L-glutamine, kept in the incubator at 36.5° C. The cells were then incubated at 36.5° C., 5% CO₂ in a static incubator for 48 hours.

### Results

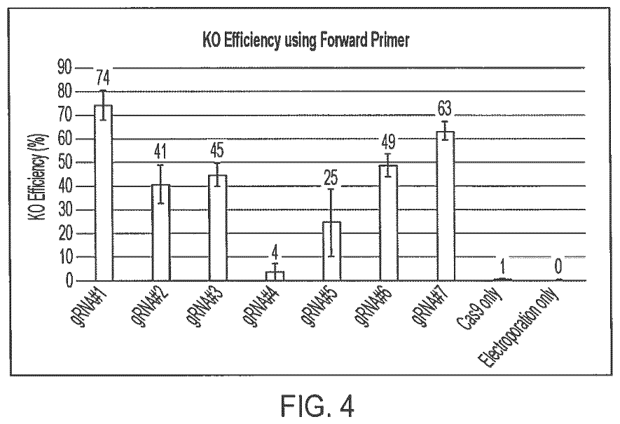

Transfection efficiency assessment performed using PCR and TIDE analysis. The assessment results are shown in FIG. 4. Based on the transfection efficiency assessment, sgRNA #1 and sgRNA #7 were selected.

### Example 2: Complete Excision of Light Chain Variable Region from Full IgG Using Cas9 RNP Complex

### Method

SSI cells expressing full IgG antibody were sub-cultured in the same manner as described in Example 1. sgRNA #1 and sgRNA #7 were selected and prepared in the same manner as described in Example 1 for nucleofection.

Cas9 RNP complexes were prepared according to Table 1.

TABLE 1
RNP complexes

| Tube | Cas9 protein | sgRNA | sgRNA | TE buffer | |
|---|---|---|---|---|---|
| No. | (μl) | (μl) | (μl) | (μl) | Nucleofection |
| 1 | 3.2 | sgRNA #1 | sgRNA #7 | — | + |
| | | 3.2 | 3.2 | | |
| 2 | 3.2 | sgRNA #1 | sgRNA #7 | 3.2 | + |
| | | 1.6 | 1.6 | | |
| 3 | 3.2 | sgRNA #1 | — | 3.2 | + |
| | | 3.2 | | | |
| 4 | 3.2 | — | sgRNA #7 | 3.2 | + |
| | | | 3.2 | | |
| 5 | 3.2 | — | — | 6.4 | + |
| 6 | — | — | — | 9.6 | + |
| 7 | — | — | — | 9.6 | − |

The complexes were incubated at room temperature for 30-60 minutes.

SSI cells expressing full IgG antibody were prepared for nucleofection by transferring 7×10⁶ cells to a new tube and centrifuged at 200 g for 5 min to remove as much media as possible. The cells were then re-suspended in 492.8 μl of Nucleofection solution (600 μl of SF solution prepared by mixing 492 μl of SF solution with 108 μl of supplement 1). 70.4 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium warmed to a 36.5° C. The entire 100 μl of the final mixtures were transferred to the 12-well plate with 1.5 ml of a CD-CHO medium, kept in the incubator at 36.5° C. The cells were then incubated at 36.5° C., 5% CO₂ in a static incubator for 48 hours.

### Results

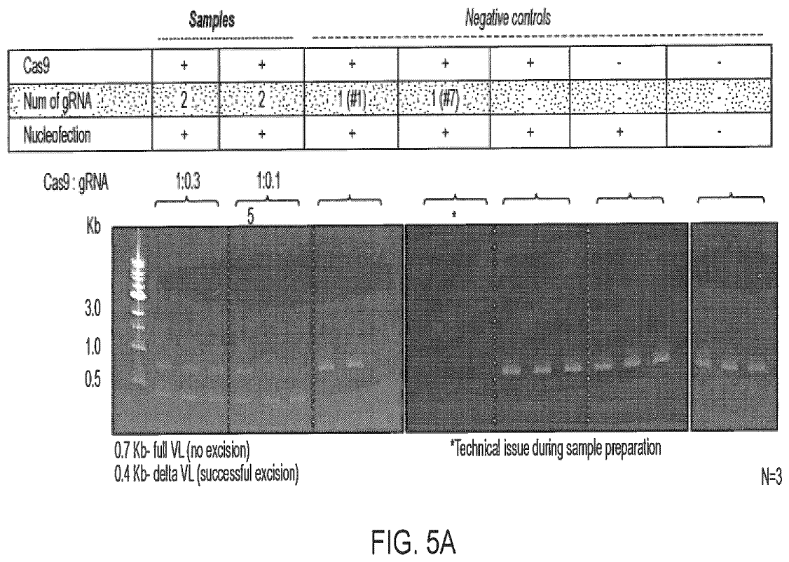

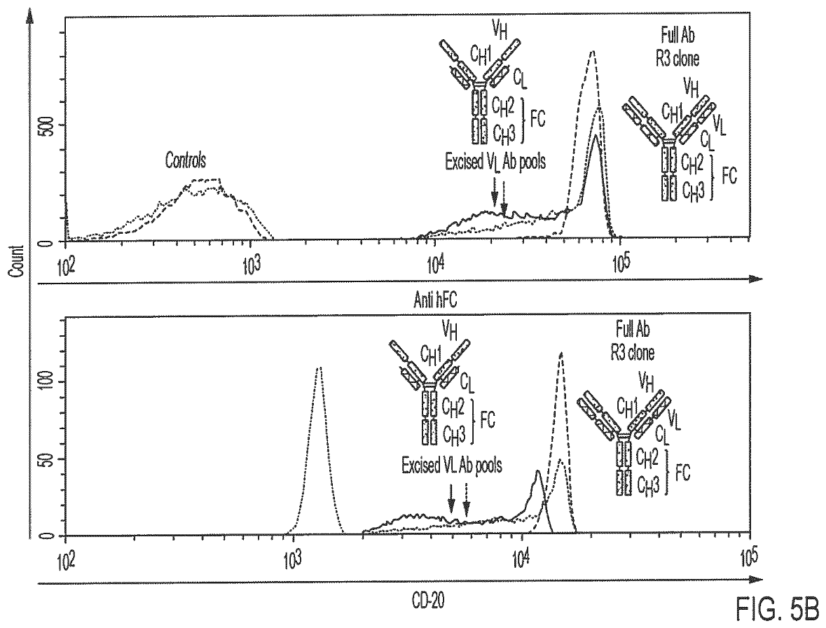

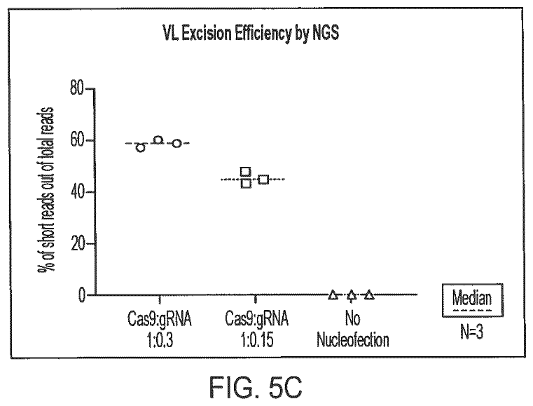

Evaluation of excision efficiency was determined using Next Generation Sequence (NGS) PCR and FACS. The results are shown in FIG. 5A-5C. PCR (FIG. 5A) FACS (FIG. 5B) and NGS (FIG. 5C) evaluation show clear and successful excision of the light chain variable region.

### Example 3: Selecting sgRNAs for Seamless Integration of Light Chain Variable Region into IgG Lacking Light Chain Variable Region (Delta VL IgG)

### Method

SSI cells expressing delta VL IgG antibody were sub-cultured in the same manner as described in Example 1. sgRNAs (TrueGuide Synthetic sgRNA 3 nmol (Invitrogen. Cat No. A35514)) were prepared in the same manner as described in Example 1 for nucleofection. Following 3 sgRNA sequences were prepared.

| | | Targeted |
|---|---|---|
| Guide name | Sequence (5′ to 3′) | DNA strand |
| sgRNA #8 | TGATCATGTCTCGGGGCAAG | + |
| (eCHO8) | (SEQ ID NO: 8) | |
| sgRNA #9 (=#6) | GAACACGGAGGGAGCGGCCA | − |
| (eCHO9) | (SEQ ID NO: 9) | |
| sgRNA #10 | GTCTCGGGGCAAGCGGACCG | + |
| (eCHO10) | (SEQ ID NO: 10) | |

Cas9 RNP complexes were prepared according to Table 2.

TABLE 2
RNP complexes

| | | Cas9 | | TE | |
|---|---|---|---|---|---|
| | | protein | sgRNA | buffer | |
| Tube No. | | (μl) | (3.2 μl) | (μl) | Nucleofection |
| 1 | Delta | 3.2 | sgRNA #8 | — | + |
| 2 | VL-T1 | 3.2 | sgRNA #9 | — | + |
| 3 | | 3.2 | sgRNA #10 | — | + |
| 4 | Delta | 3.2 | sgRNA #8 | — | + |
| 5 | VL-T2 | 3.2 | sgRNA #9 | — | + |
| 6 | | 3.2 | sgRNA #10 | — | + |
| 7 | Delta | 3.2 | — | 3.2 | + |
| 8 | VL-T1 | — | — | 6.4 | + |
| 9 | | — | — | 6.4 | + |
| 10 | Delta | 3.2 | — | 3.2 | + |
| 11 | VL-T2 | — | — | 6.4 | + |
| 12 | | — | — | 6.4 | − |

The complexes were incubated at room temperature for 30-60 minutes.

SSI cells expressing delta VL antibody (Delta VL-T1 and T2) were prepared for nucleofection by transferring 2×10⁶ cells of each cells type (delta VL T1 and T2) to a new tube and centrifuged at 200 g for 5 min to remove as much media as possible. The cells were then re-suspended in 588.8 μl of Nucleofection solution (1200 μl of SF solution prepared by mixing 984 μl of SF solution with 216 μl of supplement 1). 73.6 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium warmed to a 36.5° C. 20 μl of the final mixtures were transferred to the 96-well plate with 100 μl of a CD-CHO medium, kept in the incubator at 36.5° C. The cells were then incubated at 36.5° C., 5% CO₂ in a static incubator for 48 hours.

### Results

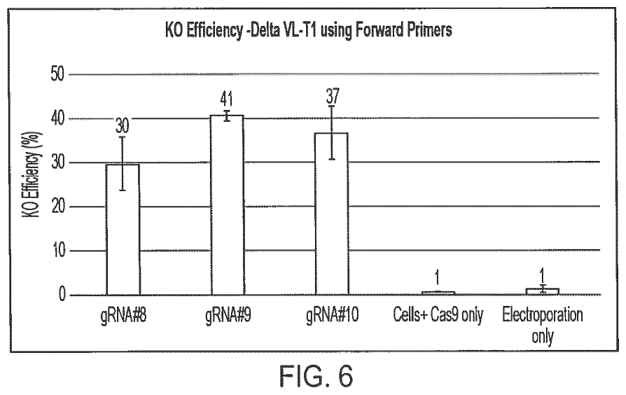

Transfection efficiency assessment performed using PCR and NGS. The assessment results are shown in FIG. 6. Based on the transfection efficiency assessment, sgRNA #9 was selected.

### Example 4: Seamless Integration of Light Chain Variable Region into IgG Lacking Light

### Chain Variable Region (Delta VL IgG)

Method

SSI cells expressing delta VL IgG antibody were sub-cultured in the same manner as described in Example 1. sgRNA #9 was selected and prepared in the same manner as described in Example 1 for nucleofection. 100 μg of light chain variable region DNA template was centrifuged briefly to collect contents and re-suspended with 25 μl of TE buffer to receive a concentration of 4 μg/μl.

Cas9 RNP complexes were prepared according to Table 3.

TABLE 3
RNP complexes

| Tube | Cas9 | sgRNA #9 | | DNA template | TE buffer | |
|---|---|---|---|---|---|---|
| No. | protein (μl) | (μl) | | (μg, μl) | (μl) | Nucleofection |
| 1 | 3.2 | 3.2 | ssSDNA | 0.4 μg (for 4 | 1.2 | + |
| | | | | samples), 2 μl | | |
| | | | | (out of 0.2 | | |
| | | | | μg/μl) | | |
| 2 | 3.2 | 3.2 | | 2 μg, 2 μl | 1.2 | + |
| | | | | (out of 1 μg/μl) | | |
| 3 | 3.2 | 3.2 | | 4 μg, 1 μl | 2.2 | + |
| 4 | 3.2 | 3.2 | | 12 μg, 3 μl | 0.2 | + |
| 5 | 3.2 | 3.2 | dsDNA | 0.4 μg (for 4 | 1.2 | + |
| | | | | samples), 2 μl | | |
| | | | | (out of 0.2 | | |
| | | | | μg/μl) | | |
| 6 | 3.2 | 3.2 | | 2 μg, 2 μl | 1.2 | + |
| | | | | (out of 1 μg/μl) | | |
| 7 | 3.2 | 3.2 | | 4 μg, 1 μl | 2.2 | + |
| 8 | 3.2 | 3.2 | | 12 μg, 3 μl | 0.2 | + |
| 9 | 3.2 | 3.2 | | — | 3.2 | + |
| 10 | 3.2 | — | ssDNA | 4 μg, 1 μl | 5.4 | + |
| 11 | — | 3.2 | | 4 μg, 1 μl | 5.4 | + |
| 12 | 3.2 | — | dsDNA | 4 μg, 1 μl | 5.4 | + |
| 13 | — | 3.2 | | 4 μg, 1 μl | 5.4 | + |
| 14 | — | — | | — | 9.6 | + |
| 15 | 3.2 | 3.2 | ssDNA | 4 μg, 1 μl | 2.2 | − |
| 16 | 3.2 | 3.2 | dsDNA | 4 μg, 1 μl | 2.2 | − |
| 17 | — | — | | — | 9.6 | − |

The complexes were incubated at room temperature for 30-60 minutes.

SSI cells expressing delta VL antibody were prepared for nucleofection by transferring 17×10⁶ cells to a new tube and centrifuged at 200 g for 5 min to remove as much media as possible. DNA templates according to Table 3 were added to the appropriate Cas9 RNP complexes as prepared. The cells were then re-suspended in 1196.8 μl of Nucleofection solution (1250 μl of SF solution prepared by mixing 1025 μl of SF solution with 225 μl of supplement 1). 70.4 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium warmed to a 36.5° C. 100 μl of the final mixtures were transferred to the 12-well plate with 1.5 ml of a CD-CHO medium, kept in the incubator at 36.5° C. The cells were then incubated at 36.5° C., 5% CO₂ in a static incubator for 48 hours.

### Results

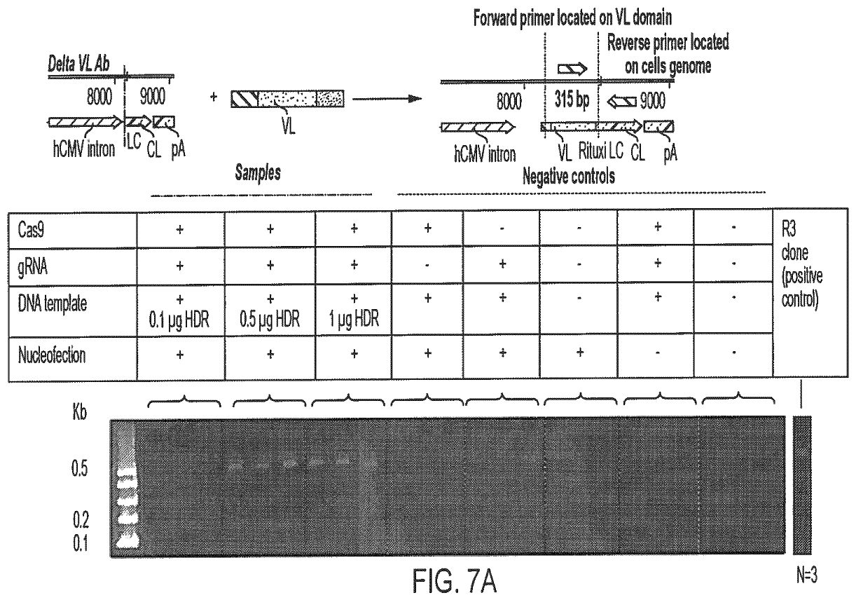

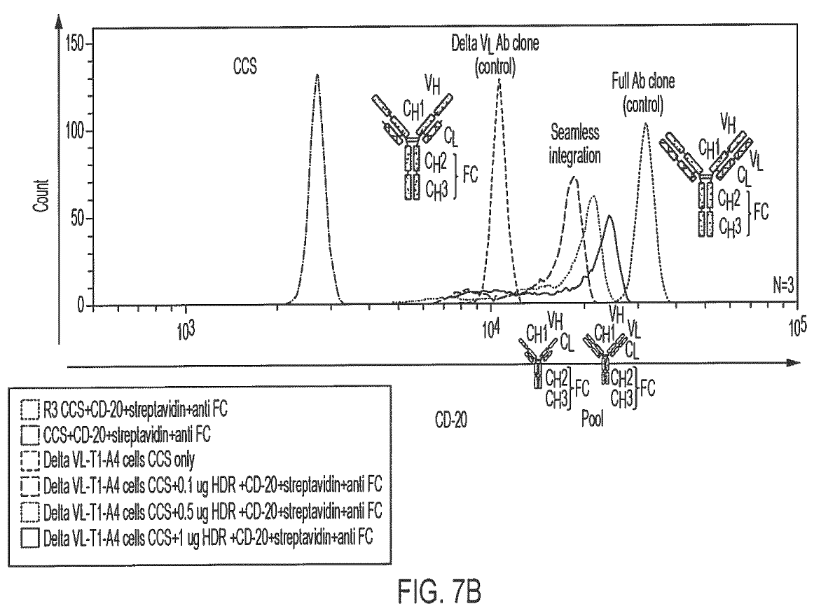

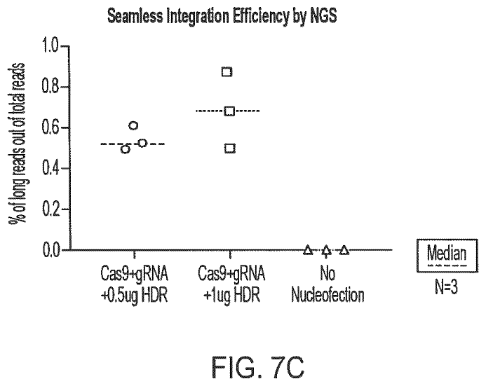

Integration efficiency assessment performed using PCR and TIDE assay. The assessment results are shown in FIGS. 7A-7C. PCR (FIG. 7A), FACS (FIG. 7B) and NGS (FIG. 7C) evaluation show clear and successful integration of the light chain variable region into delta VL antibody expressing cells.

### Example 5: Creating Inducible Cas9 Single Site Integration CHO Cells

### Method

Transduction medium was prepared by diluting Polybrene from stock solution (10 mg/mL) to a final concentration of 10 μg/mL (1:1000 dilution factor), which was divided into Tube 1 and Tube 2 with 6 mL and 3 mL, respectively. Then Edi-R inducible lentiviral hEFla-Blast-Cas9 lentiviral nuclease particles were added to Tube 2. The volume of lentiviral particles to be added was determined by the equation:

$$V = M O I \times CN \div VT \times 1000$$

where:
   V=volume of lentiviral stock in μL
   MOI=desired multiplicity of infection
   CN=number of cells in the well at transduction
   VT=lentiviral titer in TU/mL and multiplied by 1000 to convert the volume from mL to μL.

Based on the desired MOI of 1, cell density of 0.1×10⁶ cells per well at the time of transduction, and Lentiviral titer of 1×10⁷ TU/mL, 25 μL of lentiviral stock per well was calculated.

CHO cells were prepared in Tube A and Tube B at the amount of 0.6×10⁶ cells and 0.3×10⁶, respectively, and centrifuged at 200 g for 5 minutes. The cells were then resuspended with the transduction medium as prepared above so that the content of Tube 1 is added to Tube A to contain cells without lentiviral particles and Tube 2 is added to Tube B to contain cells with lentiviral particles. These cells were seeded into a 12-well plate according to plate layout of Table 4 with 1 ml in each well and incubated at 37° C. in a humidified CO₂ incubator for 24 hours.

TABLE 4
Plate layout for cell seeding

| | | |
|---|---|---|
| Well #1 | Well #4 | Well #7 |
| Tube 1 | Tube 1 | Tube 2 |
| (Untranduced) | (Untranduced + Blast) | (+LV) |
| Well #2 | Well #5 | Well #8 |
| Tube 1 | Tube 1 | Tube 2 |
| (Untranduced) | (Untranduced + Blast) | (+LV) |
| Well #3 | Well #6 | Well #9 |
| Tube 1 | Tube 1 | Tube 2 |
| (Untranduced) | (Untranduced + Blast) | (+LV) |

After 24 hours, the content of each well was centrifuged at 200 g for 5 minutes to remove the medium, and resuspended with 1 ml of pre-warmed CD-CHO/6 mM L-glutamine medium, prepared by adding 10 μL Polybrene to 10 mL CD-CHO/6 mM L-Glutamine, and seeded in a new plate according to the plate layout of Table 4 with 1 ml in each well. The cells were incubated at 37° C. in a humidified CO₂ incubator for 72 hours.

After 72 hours, selection medium containing Blasticidine in a concentration of 5 μg/mL were used to select for transduced cells. The cells were subcultured as needed and Blasticidine was added to appropriate wells at each subculturing.

### Results

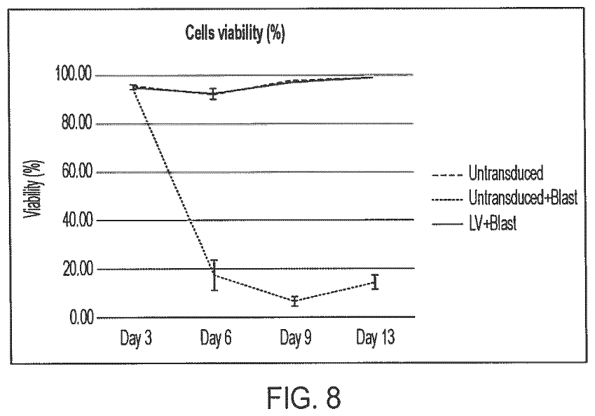

Cell viability assessment was performed using Vi-Cell device. The assessment results are shown in FIG. 8. Cell viability results clearly shows successful transduction by Edi-R inducible lentiviral hEFla-Blast-Cas9 lentiviral nuclease particles to create inducible Cas9 HD7876 Bob cells.

### Example 6: Creating Inducible Cas-SSI Cell Line Expressing IgG Antibody Lacking its VL Domain

### Method

iCas CHO host cells were sub-cultured at a viable concentration of 0.3×10⁶ cells/mL into an appropriately sized Erlenmeyer shake-flask (with CD-CHO medium supplemented with 6 mM L-glutamine). These cells have been in culture for no less than 4 days and no more than 4 weeks and have viability greater than 90% prior to transfection.

Purified delta VL (eCHO-1) plasmid containing the gene of interest and pMF35 plasmid containing an FlpE recombinase expression cassette were mixed in a DNase free sterile tube and the volume adjusted to 100 μL using TE buffer, according to Table 5.

TABLE 5
Plasmid mixture preparation

| | pEE_QGV_SSI | pMF35 | | |
|---|---|---|---|---|
| | (5 × 4 μg) | (45 μg per | TE buffer | |
| | (5 μg per 1 replica) | 1 replica) | (μl) | Electroporation |
| 1 | Delta VL (10 μg) | + (90 μg) | Up to 200 μl | + |
| 2 | Delta VL (5 μg) | + (45 μg) | Up to 100 μl | − |
| 3 | Delta VL (5 μg) | − | Up to 100 μl | + |
| 4 | — | − | 100 μl | + |
| 5 | — | − | 100 μl | − |

100 μL of plasmid mix from Table 5 were transferred to a 0.4 cm electroporation cuvette (per transfection), to which 0.8 ml of iCas CHO host cells, prepared and resuspended in 6.4 ml CD CHO/6 mM L-glutamin medium, were added. The cells were electroporated by delivering a single exponential decay pulse of 300 V, 900 μF.

The contents of the cuvette were transferred to each of the 20 mL of CD CHO/6 mM L-glutamine medium added to six T75 flasks and pre-warmed to 36.5° C. in a humidified static CO₂ incubator. The flasks were incubated for 24 hours in the humidified static CO₂ incubator.

After 24 hours, the medium of each flask was replaced with 20 ml pre-warmed CD CHO medium. The cells were cultured and subcultured.

### Results

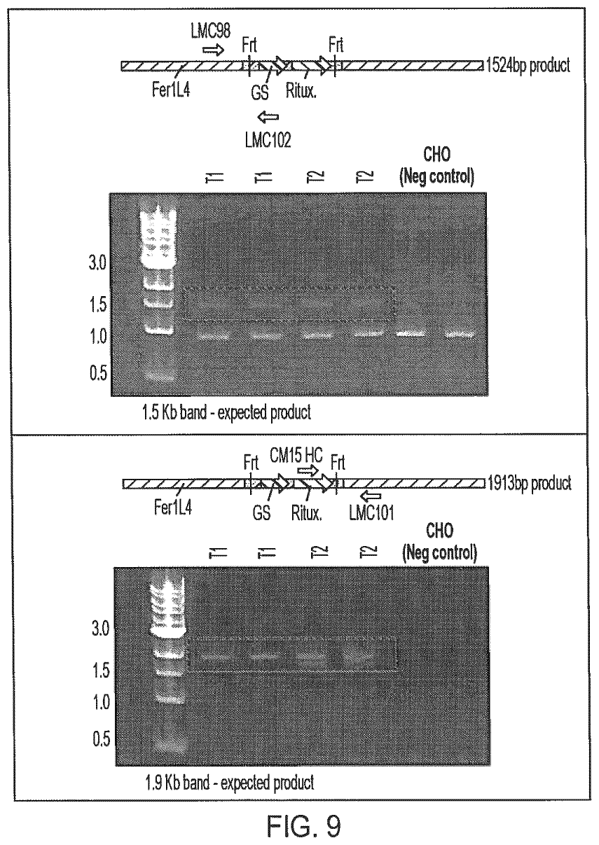

Transfection efficiency assessment performed using PCR. The assessment results are shown in FIG. 9. The PCR assessment shows clear and successful integration of delta VL into iCas HD7876 Bob host cells to create inducible Cas-SSI cell line.

### Example 7: Seamless Integration of Light Chain Variable Region into Inducible Cas-SSI Cell Line Expressing IgG Antibody Lacking its VL Domain

### Method

SSI cells expressing delta VL IgG antibody were sub-cultured at a viable concentration of 0.3×10⁶ cells/mL into an appropriately sized Erlenmeyer shake-flask (CD-CHO medium and Blasticidin in a final concentration of 5 μg/mL). One day prior to transfection, Doxycycline of final concentration of 2.5 μg/mL was added to the cell culture. sgRNA #9 was selected and prepared in the same manner as described in Example 1 for nucleofection. 100 μg of light chain variable region DNA template was centrifuged briefly to collect contents and re-suspended with 25 μl of TE buffer to receive a concentration of 4 μg/μl.

Cas9 RNP complexes were prepared according to Table 6.

TABLE 6
RNP complexes

| Tube | Cas9 | sgRNA #9 | | DNA template | TE buffer | |
|---|---|---|---|---|---|---|
| No. | protein (μl) | (μl) | | (μg, μl) | (μl) | Nucleofection |
| 1 | 3.2 | 3.2 | dsDNA | 0.4 μg (for 4 | 1.2 | + |
| | | | | samples), 2 μl | | |
| | | | | (out of 0.2 μg/μl) | | |
| 2 | 3.2 | 3.2 | | 2 μg, 2 μl | 1.2 | + |
| | | | | (out of 1 μg/μl) | | |
| 3 | 3.2 | 3.2 | | 4 μg, 1 μl | 2.2 | + |
| 4 | 3.2 | — | | 4 μg, 1 μl | 5.4 | + |
| 5 | — | 3.2 | | 4 μg, 1 μl | 5.4 | + |
| 6 | — | — | | — | 9.6 | + |
| 7 | 3.2 | 3.2 | | 4 μg, 1 μl | 2.2 | − |
| 8 | — | — | | — | 9.6 | − |

The complexes were incubated at room temperature for 30-60 minutes.

SSI cells expressing delta VL antibody were prepared for nucleofection by transferring 8×10⁶ cells to a new tube and centrifuged at 200 g for 5 min to remove as much media as possible. DNA templates according to Table 6 were added to the appropriate Cas9 RNP complexes as prepared. The cells were then re-suspended in 563.2 μl of Nucleofection solution (600 μl of SF solution prepared by mixing 492 μl of SF solution with 108 μl of supplement 1). 70.4 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium containing Doxycycline in a final concentration of 2.5 μg/mL warmed to a 36.5° C. 100 μl of the final mixtures were transferred to the 12-well plate with 1.5 ml of a CD-CHO medium containing Blasticidin in a final concentration of 5 μg/mL, kept in the incubator at 36.5° C. The cells were then incubated at 36.5° C., 5% CO₂ in a static incubator for 48 hours.

### Results

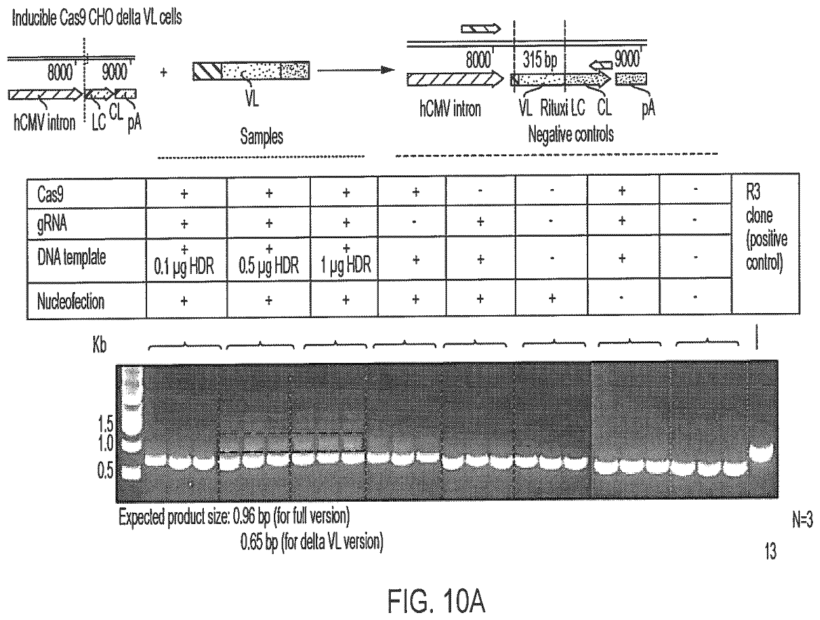

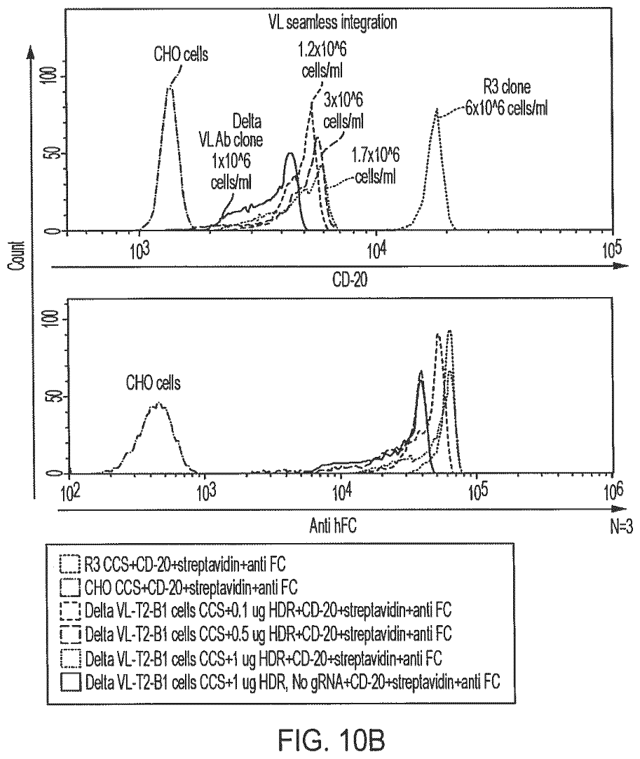

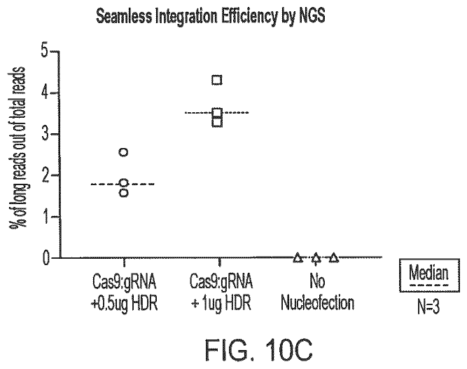

Integration efficiency assessment performed using PCR and TIDE assay. The assessment results are shown in FIGS. 10A-10C. PCR (FIG. 10A), FACS (FIG. 10B) and NGS (FIG. 10C) evaluation show clear and successful integration of the light chain variable region into delta VL antibody expressing cells.

### Example 8: Seamless Integration of Heavy Chain Variable Region and Light Chain Variable Region Into Headless IgG Antibody

### Method

SSI cells expressing headless IgG antibody are sub-cultured at a viable concentration of 0.3×10⁶ cells/mL into an appropriately sized Erlenmeyer shake-flask (CD-CHO medium and Blasticidin in a final concentration of 5 μg/mL). One day prior to transfection, Doxycycline of final concentration of 2.5 μg/mL is added to the cell culture. sgRNA #9 is selected and prepared for nucleofection. 100 μg of DNA template that consist of both VL domain and VH domain is centrifuged briefly to collect contents and re-suspended with 25 μl of TE buffer to receive a concentration of 4 μg/μl.

Cas9 RNP complexes are prepared according to Table 7.

TABLE 7
RNP complexes

| Tube | Cas9 | sgRNA #9 | | DNA template | TE buffer | |
|---|---|---|---|---|---|---|
| No. | protein (μl) | (μl) | | (μg, μl) | (μl) | Nucleofection |
| 1 | 3.2 | 3.2 | ssDNA | 0.4 μg (for 4 | 1.2 | + |
| | | | | samples), 2 μl | | |
| | | | | (out of 0.2 | | |
| | | | | μg/μl) | | |
| 2 | 3.2 | 3.2 | | 2 μg, 2 μl | 1.2 | + |
| | | | | (out of 1 μg/μl) | | |
| 3 | 3.2 | 3.2 | | 4 μg, 1 μl | 2.2 | + |
| 4 | 3.2 | 3.2 | | 12 μg, 3 μl | 0.2 | + |
| 5 | 3.2 | 3.2 | Stable- | 0.4 μg (for 4 | 1.2 | + |
| | | | dsDNA | samples), 2 μl | | |
| | | | | (out of 0.2 | | |
| | | | | μg/μl) | | |
| 6 | 3.2 | 3.2 | | 2 μg, 2 μl | 1.2 | + |
| | | | | (out of 1 μg/μl) | | |
| 7 | 3.2 | 3.2 | | 4 μg, 1 μl | 2.2 | + |
| 8 | 3.2 | 3.2 | | 12 μg, 3 μl | 0.2 | + |
| 9 | 3.2 | 3.2 | | — | 3.2 | + |
| 10 | 3.2 | — | ssDNA | 4 μg, 1 μl | 5.4 | + |
| 11 | — | 3.2 | | 4 μg, 1 μl | 5.4 | + |
| 12 | 3.2 | — | Stable- | 4 μg, 1 μl | 5.4 | + |
| 13 | — | 3.2 | dsDNA | 4 μg, 1 μl | 5.4 | + |
| 14 | — | — | | — | 9.6 | + |
| 15 | 3.2 | 3.2 | ssDNA | 4 μg, 1 μl | 2.2 | − |
| 16 | 3.2 | 3.2 | Stable- | 4 μg, 1 μl | 2.2 | − |
| | | | dsDNA | | | |
| 17 | — | — | | — | 9.6 | − |

The complexes are incubated at room temperature for 30-60 minutes.

SSI cells expressing headless IgG antibody are prepared for nucleofection by transferring 8×10⁶ cells to a new tube and centrifuged at 200 g for 5 min to remove as much media as possible. DNA templates according to Table 7 are added to the appropriate Cas9 RNP complexes as prepared. The cells are then re-suspended in 563.2 μl of Nucleofection solution (600 μl of SF solution prepared by mixing 492 μl of SF solution with 108 μl of supplement 1). 70.4 μl of cells are added to each of the Cas9 RNP complex mixtures.

20 μl of the cells-nucleofection solution-RNP complex mixture are transferred to a cuvette in the nucleofection 16-well strip where cells are pulsed using the Lonza 4D nucleofector unit using program DU-158. The cells are left to sit in the nucleofection cuvettes for 10 min post nucleofection and added and mixed 80 μl of pre-warmed 10 ml of CD-CHO medium containing Doxycycline in a final concentration of 2.5 μg/mL warmed to a 36.5° C. 100 μl of the final mixtures are transferred to the 12-well plate with 1.5 ml of a CD-CHO medium containing Blasticidin in a final concentration of 5 μg/mL, kept in the incubator at 36.5° C. The cells are then incubated at 36.5° C., 5% CO₂ in a static incubator for 48 hours.

### Results

Integration efficiency assessment is performed using PCR, NGS, FACS analysis, and Western Blot analysis.

### General Material and Methods

### SDS-PAGE and Western Blotting

Cell culture supernatant (CCS) was collected and centrifuged at 3,500×g for 10 minutes. Protein concentration was determined using the BCA Protein Assay Kit (Sigma Aldrich #71285-3). Protein extracts (30 μg) were mixed with 4× Laemmli protein sample buffer (Biorad #1610747) incubated at 75° C., for 15 minutes, and resolved according to their molecular weight by MP TGX 4-20% 12 well SDS page gel in Tris/Glycine/SDS Buffer ×1 (Biorad #4561095) using Mini-PROTEAN Tetra Vertical Electrophoresis Cell (Biorad #1658004). The gels were run at room temperature at 120V until the appropriate separation was achieved.

Proteins were then transferred onto a nitrocellulose membrane using Trans-Blot Turbo Transfer System (Biorad #1704158). Membranes were then blocked in 5% BSA in PBS-T (0.1% Tween) for 1 hour at room temperature and then incubated with the indicated antibody (Goat Anti-Human IgG Fc (HRP) ab97225) diluted in 5% BSA in PBS-T for 1 hr at room temperature. Next, the membranes underwent 3-5 washes with PBS supplemented with 0.2% Tween. Finally, the membranes were developed using Immobilon Forte Western HRP substrate (SigmaAldrich #WBLUF0100) using a bright Imaging System (Rhenium).

### DNA Extraction and PCR Amplification

Genomic DNA was extracted using Quick Extract DNA Extraction Solution (Danyel Biotech #QE09050) according to manufacturer's instructions, quantified using NanoDrop One/OneC Microvolume UV-Vis Spectrophotometer (ThermoFisher) with 100 ng/PCR reaction. Primers were designed using SnapGene with ˜50% CG content and melting temperatures below 60° C. (Table 8). PCR was performed using a Phusion® Hot Start Flex 2× Master Mix (New England Biolabs #M0536S) following the manufacturer's instructions with the Proflex PCR system (ThermoFisher Scientific). PCR products were loaded into 1% agarose gel (1% of Agarose (SigmaAldrich #A9539) in 1×TAE (Biological industries #18701A) and electrophoresis was performed.

TABLE 8
PCR primers

| Primer Name | Sequence (5′-3′) |
|---|---|
| TIDE-F1 | taactcccgttgcggtgctg |
| | (SEQ ID NO: 11) |
| TIDE-R1 | ggaggacaggctgtaggtgc |
| | (SEQ ID NO: 12) |
| P1-Frw | gtctgaaaatgagctcgggg |
| | (SEQ ID NO: 13) |
| VH-R for TIDE | ggcttggtcttggcgttgt |
| | (SEQ ID NO: 14) |
| CM15_HC_F | CCTGATCCTGCTGTTCCTGG |
| | (SEQ ID NO: 15) |
| CM15_-HC_R | CCCTAGGTCATCACTTGCCG |
| | (SEQ ID NO: 16) |
| LMC98 | GGAGTTCAAAGCCAGCTTATACCAACATG |
| | (SEQ ID NO: 17) |
| LMC101 | CCTCTTCTTCAGCCACAGTCACTTC |
| | (SEQ ID NO: 18) |
| LMC102 | GCTAAAGTTGGTATGGCAGCCTGCACC |
| | (SEQ ID NO: 19) |
| Ritux_FRT_F | CCAAGCCCAGAGAGGAACAG |
| | (SEQ ID NO: 20) |
| Ritux_FRT_R | GACAGGTATCCGGTAAGCGG |
| | (SEQ ID NO: 21) |
| Ritux_F5_F | TTCATTCAGCTCCGGTTCCC |
| | (SEQ ID NO: 22) |
| Ritux_F5_R | GTGCCCATCTGTTCCCATCA |
| | (SEQ ID NO: 23) |
| P1 Rev for TIDE | tgaattcatcagcactcgcc |
| | (SEQ ID NO: 24) |
| VH-KIMN | caccagctacaacatgcact |
| | (SEQ ID NO: 25) |
| Reverse MN | cggagcaggagaacacgttg |
| | (SEQ ID NO: 26) |
| VL-KI MN | ctggatctacgccacctcca |
| | (SEQ ID NO: 27) |
| Rev for VL | ggcggccgccagacatgata |
| insertion | (SEQ ID NO: 28) |
| CL-D2-F1 | gttgaaggacttggtcacgg |
| | (SEQ ID NO: 29) |
| VH-R | agtctttgaccaggcagccc |
| | (SEQ ID NO: 30) |
| SP-F | gatgatctgcacctggaagt |
| | (SEQ ID NO: 31) |
| mCMV-R1 | catCGTTctggtactgagtc |
| | (SEQ ID NO: 32) |

### Flow Cytometry

Cells/cells culture supernatant (CCS) were examined by flow cytometry electroporation to determine cell line generation, VL/VL excision and integration efficiencies. All flow cytometry experiments were performed on the CytoFLEX S Flow Cytometer (Beckman coulter) and CytExpert 2.4 software were used for analyzing the acquired immunofluorescence data.

For cell line generation verification, cells were collected in 96-well U shaped microplates, washed twice in FACS Buffer (2% w/v Bovine Serum Albumin (SigmaAldrich #810533) and 0.09% v/v Sodium Azide (SigmaAldrich #08591) in DPBS), centrifuged at 400 rpm for 3 minutes, and analyzed using FACS. GFP positive cells were detected using the GFP channel (488/525-40) on the CytoFLEX S Flow Cytometer.

CCS was collected from cells at concentration of 5 million cells/mL, centrifuged at 3500×g for 10 min, and freezed at (−80° C.). For excision and integration, 0.5 μl/well of Dynabeads Protein A for Immunoprecipitation (Invitrogen, Cat No.: 10001D) were added to each 96-well plate and washed twice with Assay buffer (PBS with 0.1% BSA, 0.2 μM filtered). 200 μl of CCS was added to each well, incubated for 30 min at room temperature using gentle rotation, and washed trice with assay buffer. Afterwards, 0.525 μg/well of Biotinylated Human CD20 Protein, His, Avitag (Acro Biosystems, Cat No.: CD0-H82E5) was added in assay buffer, incubated for 30 minutes at room temperature using gentle rotation and washed trice with assay buffer. Then, Alexa Flour 488-conjugated streptavidin Cat #016-540-084 was diluted 1:50 in each well using assay buffer, incubated for 30 minutes at room temperature using gentle rotation protected from light. Then, Alexa Fluor 647conjugated Affinity Pure F (ab) 2 Fragment Goat anti-Human IgG (H+L) (Jackson Immune Research laboratory, Cat No.: 109-606-088) was diluted to 1:250 in each well using assay buffer, incubated for 30 minutes at room temperature using gentle rotation protected from light. Lastly, beads were resuspended in 100 μl of assay buffer and analyzed using FACS. AF488 was detected using 488/525-40 filter and AF647 was detected using 630/660-20 filter on the CytoFLEX S Flow Cytometer.

### Example 9: Selecting sgRNAs for Excision of Light Chain Variable Region

### Method

SSI Cells Sub-Culturing and sgRNA Preparation

Site Specific Integration (SSI) cells (see, e.g., WO 2020/072480) expressing a full IgG antibody were sub-cultured at a viable concentration of 0.3×10⁶ cells/mL into an appropriately sized Erlenmeyer shake-flask (with CD-CHO medium). These cells were in culture for no less than 4 days and no more than 4 weeks and have viability greater than 90% prior to transfection.

sgRNAs (TrueGuide Synthetic sgRNA 3 nmol (Invitrogen. Cat No. A35514)) were centrifuged briefly to collect contents and re-suspended to 100 μM in nuclease-free Tris-EDTA (TE) buffer to generate a stock solution. A 10 μM working solution was generated from this stock. All sgRNA solutions were stored at −20° C. The following 7 sgRNA sequences were prepared.

| Guide name | Sequence (5′ to 3′) |
|---|---|
| sgRNA #1 (eCHO1) | GCCCCGAGACATGATCACGG |
| | (SEQ ID NO: 1) |
| sgRNA #2 (eCHO2) | CCGCCTCCGTGATCATGTCT |
| | (SEQ ID NO: 2) |
| sgRNA #3 (eCHO3) | GCCTCCGTGATCATGTCTCG |
| | (SEQ ID NO: 3) |
| sgRNA #4 (eCHO4) | CTTCGGCGGAGGCACCAAGC |
| | (SEQ ID NO: 4) |
| sgRNA #5 (eCHO5) | GCACCAAGCTGGAAATCAAG |
| | (SEQ ID NO: 5) |
| sgRNA #6 (eCHO6) | GAACACGGAGGGAGCGGCCA |
| | (SEQ ID NO: 6) |
| sgRNA #7 (eCHO7) | GCTGGAAATCAAGCGGACCG |
| | (SEQ ID NO: 7) |

### Nucleofection

The general nucleofection conditions, per individual transfection, were as follows:

| | |
|---|---|
| Amount of Cell | 2.5 × 10\hat{ }5 cells |
| TrueCut Cas9 Protein V2 | 4 ug (0.8 ul) |
| (Invitrogen. Cat No. A36499) | |
| sgRNA 10 uM | 0.8 ul (Cas9:sgRNA ratio is 1:0.3) |
| Nucleofection solution | SF solution supplemented |
| | with supplement 1 (82% v/v |
| | SF solution, 18% v/v supplement 1); |
| | 18.4 ul per transfection. |

Cas9 RNP complex mixtures for each sgRNA sequences above were prepared in 7 Eppendorf tubes by mixing 3.2 μl of Cas9 protein with 3.2 μl of 10 μM sgRNA. 3 control mixtures were also prepared with 1 Eppendorf tube containing 3.2 μl of Cas9 protein and 3.2 μl of TE buffer and 2 Eppendorf tubes each containing 6.4 μl of TE buffer. The mixtures were incubated at room temperature for 30-60 minutes.

SSI cells expressing full IgG antibody were prepared for nucleofection by transferring 10⁷ cells to a new tube and centrifuged at 300 g for 5 min to remove as much media as possible. The cells were then re-suspended in 736 μl of Nucleofection solution (656 μl of SF solution with 144 μl of supplement 1). 73.6 μl of cells were added to each of the 10 Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium supplemented with 6 mM L-glutamine warmed to a 36.5° C. 20 μl of the final mixtures were transferred to the 96-well plate with 100 μl of a CD-CHO medium supplemented with 6 mM L-glutamine, kept in the incubator at 36.5° C. The cells were then incubated at 36.5° C., 5% CO₂ in a static incubator for 48 hours.

### Results

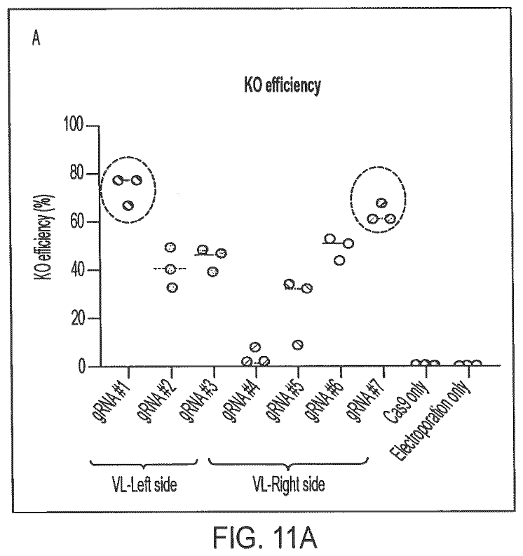

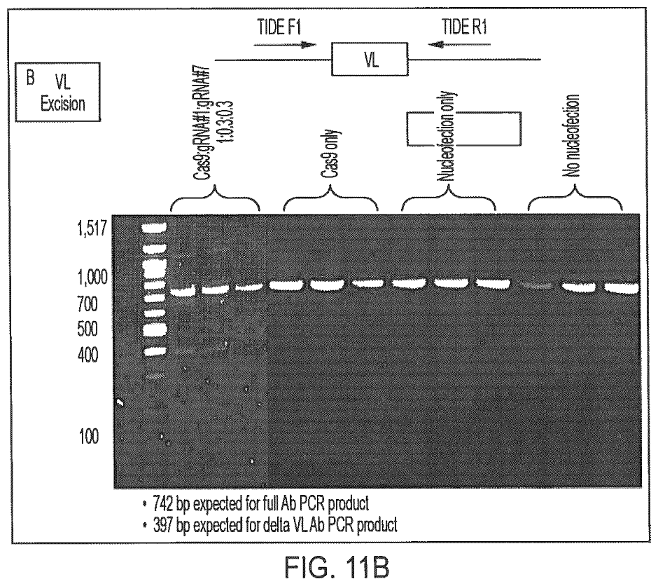

Transfection efficiency assessment performed using TIDE analysis. The assessment results are shown in FIG. 11A. Based on the transfection efficiency assessment, sgRNA #1 and sgRNA #7 were selected.

### Example 10: Complete Excision of Light Chain Variable Region from Full IgG Using Cas9 RNP Complex

### Method

SSI cells expressing full IgG antibody were sub-cultured in the same manner as described in Example 9. sgRNA #1 and sgRNA #7 were selected and prepared in the same manner as described in Example 9 for nucleofection.

Cas9 RNP complexes were prepared according to Table 9.

TABLE 9
RNP complexes

| | Cas9 | | | TE | |
|---|---|---|---|---|---|
| | protein | sgRNA | sgRNA | buffer | |
| Tube No. | (μl) | (μl) | (μl) | (μl) | Nucleofection |
| 1 | 3.2 | sgRNA #1 | sgRNA #7 | — | + |
| | | 3.2 | 3.2 | | |
| 2 | 3.2 | sgRNA #1 | sgRNA #7 | 3.2 | + |
| | | 1.6 | 1.6 | | |
| 3 | 3.2 | sgRNA #1 | — | 3.2 | + |
| | | 3.2 | | | |
| 4 | 3.2 | — | sgRNA #7 | 3.2 | + |
| | | | 3.2 | | |

The complexes were incubated at room temperature for 30-60 minutes.

SSI cells expressing full IgG antibody were prepared for nucleofection by transferring 7×10⁶ cells to a new tube and centrifuged at 200 g for 5 min to remove as much media as possible. The cells were then re-suspended in 492.8 μl of Nucleofection solution (600 μl of SF solution prepared by mixing 492 μl of SF solution with 108 μl of supplement 1). 70.4 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed with 80 μl of pre-warmed 10 ml of CD-CHO medium warmed to a 36.5° C. The entire 100 μl of the final mixtures were transferred to the 12-well plate with 1.5 ml of a CD-CHO medium, kept in the incubator at 36.5° C. The cells were then incubated at 36.5° C., 5% CO₂ in a static incubator for 48 hours.

### Results

Evaluation of excision efficiency was determined using PCR analysis. The results are shown in FIG. 11B. PCR evaluation shows clear and successful excision of the light chain variable region.

### Example 11: Selecting sgRNAs for Excision of Heavy Chain Variable Region

### Method

SSI Cells Sub-Culturing and sgRNA Preparation

Site Specific Integration (SSI) cells (see, e.g., WO 2020/072480) expressing a full IgG antibody were sub-cultured at a viable concentration of 0.3×10⁶ cells/mL into an appropriately sized Erlenmeyer shake-flask (with CD-CHO medium). These cells were in culture for no less than 4 days and no more than 4 weeks and have viability greater than 90% prior to transfection.

sgRNAs (TrueGuide Synthetic sgRNA 3 nmol (Invitrogen. Cat No. A35514)) were centrifuged briefly to collect contents and re-suspended to 100 μM in nuclease-free Tris-EDTA (TE) buffer to generate a stock solution. A 10 μM working solution was generated from this stock. All sgRNA solutions were stored at −20° C. The following 6 sgRNA sequences were prepared.

| Guide name | Sequence (5′ to 3′) |
|---|---|
| sgRNA #11 (eCHO1) | cgtgtctgccgcttccacca |
| | (SEQ ID NO: 33) |
| sgRNA #12 (eCHO2) | gggggccagagggaacacgg |
| | (SEQ ID NO: 34) |
| sgRNA #13 (eCHO3) | gaacacggaggggcccttgg |
| | (SEQ ID NO: 35) |
| sgRNA #14 (eCHO4) | gaccaggaacagcaggatca |
| | (SEQ ID NO: 36) |
| sgRNA #15 (eCHO5) | cctggtcgccgtggccaccc |
| | (SEQ ID NO: 37) |
| sgRNA #16 (eCHO6) | cccgggtggccacggcgacc |
| | (SEQ ID NO: 38) |

### Nucleofection

The general nucleofection conditions, per individual transfection, were as follows:

| | |
|---|---|
| Amount of Cell | 2.5 × 10\hat{ }5 cells |
| TrueCut Cas9 Protein V2 | 4 ug (0.8 ul) |
| (Invitrogen. Cat No. A36499) | |
| sgRNA 10 uM | 0.8 ul (Cas9:sgRNA ratio is 1:0.3) |
| Nucleofection solution | SF solution supplemented |
| | with supplement 1 (82% v/v SF solution, |
| | 18% v/v supplement 1); |
| | 18.4 ul per transfection. |

Cas9 RNP complex mixtures for each sgRNA sequences above were prepared in 6 Eppendorf tubes by mixing 3.2 μl of Cas9 protein with 3.2 μl of 10 μM sgRNA. 3 control mixtures were also prepared with 1 Eppendorf tube containing 3.2 μl of Cas9 protein and 3.2 μl of TE buffer and 2 Eppendorf tubes each containing 6.4 μl of TE buffer. The mixtures were incubated at room temperature for 30-60 minutes.

SSI cells expressing full IgG antibody were prepared for nucleofection by transferring 10⁷ cells to a new tube and centrifuged at 300 g for 5 min to remove as much media as possible. The cells were then re-suspended in 736 μl of Nucleofection solution (656 μl of SF solution with 144 μl of supplement 1). 73.6 μl of cells were added to each of the 10 Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed with 80 μl of pre-warmed 10 ml of CD-CHO medium warmed to a 36.5° C. 20 μl of the final mixtures were transferred to the 96-well plate with 100 μl of a CD-CHO medium, kept in the incubator at 36.5° C. The cells were then incubated at 36.5° C., 5% CO₂ in a static incubator for 48 hours.

### Results

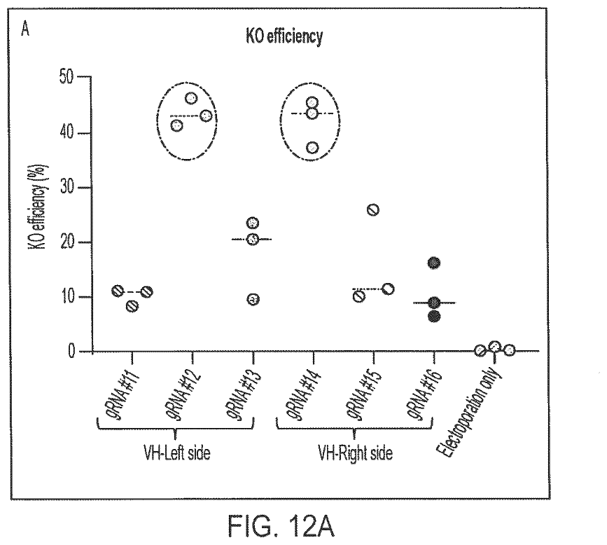

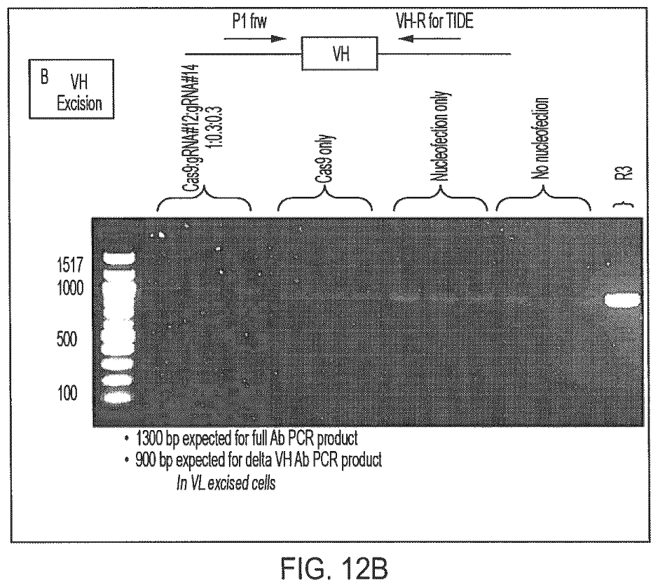

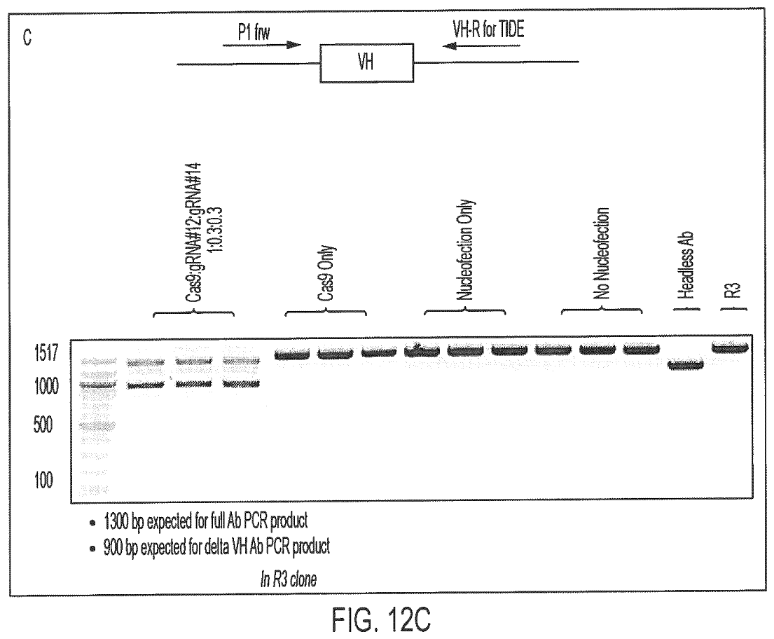

Transfection efficiency assessment performed using TIDE analysis. The assessment results are shown in FIG. 12A. Based on the transfection efficiency assessment, sgRNA #12 and sgRNA #14 were selected.

### Example 12: Complete Excision of Heavy Chain Variable Region from Full IgG or Light Chain Variable Region Excised Cells (Delta VL Cells) Using Cas9 RNP Complex

Method

SSI cells expressing full IgG antibody and VL excised cells (delta VL) were sub-cultured in the same manner as described in Example 11. sgRNA #12 and sgRNA #14 were selected and prepared in the same manner as described in VH excision gRNAs assessments for nucleofection.

Cas9 RNP complexes were prepared according to Table 10.

TABLE 10
RNP complexes

| | Cas9 | | | TE | |
|---|---|---|---|---|---|
| | protein | sgRNA | sgRNA | buffer | |
| Tube No. | (μl) | (μl) | (μl) | (μl) | Nucleofection |
| 1 | 3.2 | sgRNA #12 | sgRNA | — | + |
| | | 3.2 | #14 | | |
| | | | 3.2 | | |
| 2 | 3.2 | — | — | 6.4 | + |
| 3 | — | — | — | 9.6 | + |
| 4 | — | — | — | 9.6 | − |

The complexes were incubated at room temperature for 30-60 minutes.

SSI cells were prepared for nucleofection by transferring 7×10⁶ cells to a new tube and centrifuged at 200 g for 5 min to remove as much media as possible. The cells were then re-suspended in 492.8 μl of Nucleofection solution (600 μl of SF solution prepared by mixing 492 μl of SF solution with 108 μl of supplement 1). 70.4 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium warmed to a 36.5° C. The entire 100 μl of the final mixtures were transferred to the 12-well plate with 1.5 ml of a CD-CHO medium, kept in the incubator at 36.5° C. The cells were then incubated at 36.5° C., 5% CO₂ in a static incubator for 48 hours.

### Results

Evaluation of excision efficiency was determined using PCR analysis. The results are shown in FIG. 12B-12C. PCR evaluation of delta VL cells (FIG. 12B) and SSI cells expressing full IgG antibody (FIG. 12C) show clear and successful excision of the heavy chain variable region from both cells.

### Clone Selection

Delta VH and VL cells were filtered through 40 μm nylon mesh into 5 mL round bottom tubes (Corning, Corning, NY, USA) immediately prior to sorting. Cells were gated according to FCS and SSC in FACS ArialI using 100 um nozzle and went through single cell sorting in 96 well plate.

### Example 13: Seamless Integration of Heavy Chain Variable Region into IgG Lacking Heavy and Light Chain Variable Region (Delta VH VL IgG)

### Method

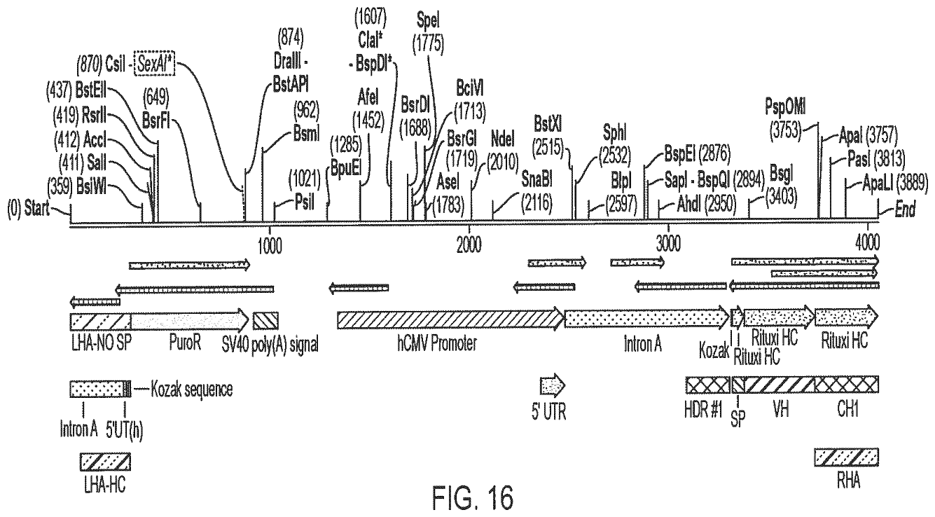

SSI cells expressing delta VH VL IgG antibody were sub-cultured in the same manner as before. sgRNA #18 with the following sequence (5′ to 3′): AGACAGCACCCGGGTGGCCA (SEQ ID NO: 39), was selected and prepared in the same manner as described before for nucleofection. 100 μg of heavy chain variable region DNA template was centrifuged briefly to collect contents and re-suspended with 25 μl of TE buffer to receive a concentration of 4 μg/μl. FIG. 16 shows representation of DNA template used for heavy chain variable region-Puromycin integration.

Cas9 RNP complexes were prepared according to Table 11.

TABLE 11
RNP complexes

| Tube | Cas9 | sgRNA #18 | | DNA template | TE buffer | |
|---|---|---|---|---|---|---|
| No. | protein (μl) | (μl) | | (μg, μl) | (μl) | Nucleofection |
| 1 | 3.2 | 3.2 | Stable | 1.6 μg (for 4 | 1.2 | + |
| | | | dsDNA | samples), | | |
| | | | | 2 μl 160 fmol | | |
| | | | | (out of 0.8 μg/μl) | | |
| 2 | 3.2 | 3.2 | | 4 μg, 2 μl 400 fmol | 1.2 | + |
| | | | | (out of 2 μg/μl) | | |
| 3 | 3.2 | 3.2 | | 8 μg, 2 μl 800 fmol | 1.2 | + |
| 4 | 3.2 | — | | 8 μg, 2 μl | 4.4 | + |
| 5 | — | 3.2 | | 8 μg, 2 μl | 4.4 | + |
| 6 | — | — | | — | 9.6 | + |
| 7 | 3.2 | 3.2 | | 8 μg, 2 μl | 1.2 | − |
| 8 | — | — | | — | 9.6 | − |

SSI cells expressing delta VH VL IgG antibody were prepared for nucleofection by transferring 8×10⁶ cells to a new tube and centrifuged at 200 g for 5 min to remove as much media as possible. The cells were then re-suspended in 563.2 μl of Nucleofection solution (600 μl of SF solution prepared by mixing 492 μl of SF solution with 108 μl of supplement 1). 70.4 μl of cells were added to each of the Cas9 RNP complex mixtures.

220 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium warmed to a 36.5° C. The entire 100 μl of the final mixtures were transferred to the 12-well plate with 1.5 ml of a CD-CHO medium, kept in the incubator at 36.5° C. The cells were then incubated at 36.5° C., 5% CO₂ in a static incubator for 24 hours.

24 hours later, medium was replaced with CD-CHO+6 μg/mL Puro medium for selection. Cells were grown in static incubator until negative control were died. Cell viability assessment was performed using Vi-Cell device.

### Results

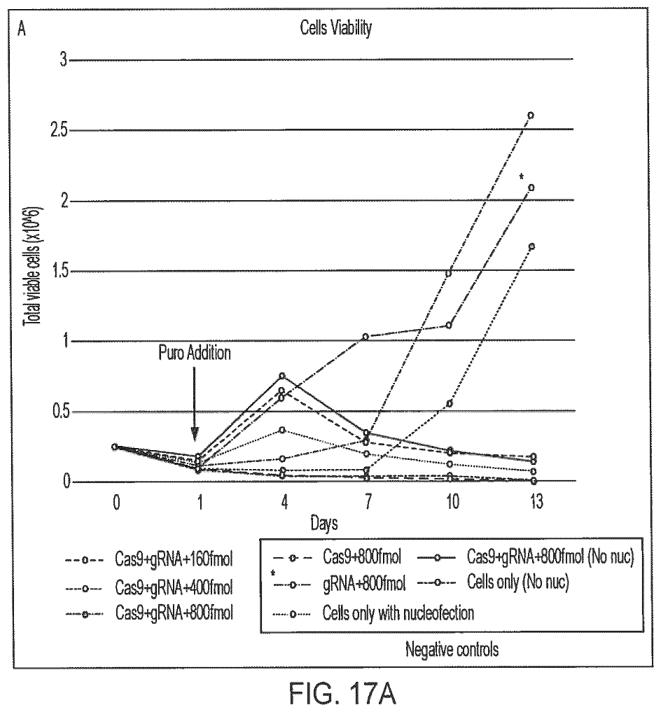

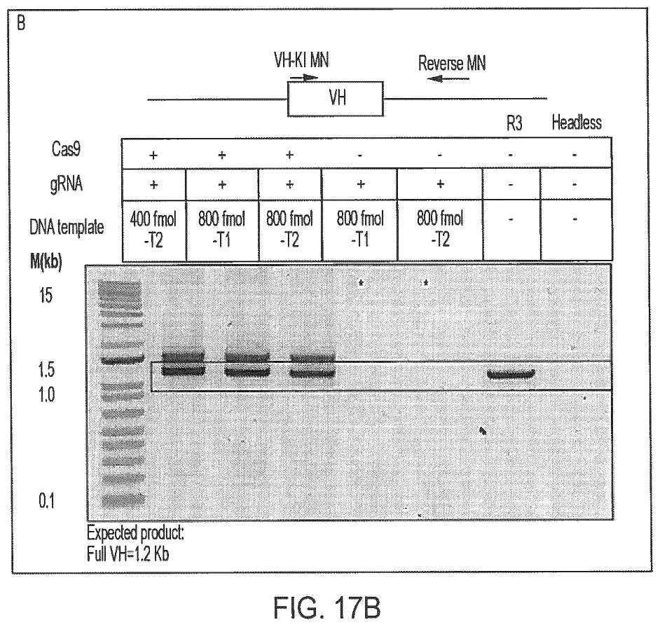

Integration of heavy chain variable region validated by PCR. Cell selection and viability assessed through Puromycin selection as shown in FIG. 17A. PCR evaluation (FIG. 17B) shows seamless integration of heavy chain variable region.

### Example 14: Seamless Integration of Light Chain Variable Region into IgG Lacking Light Chain Variable Region (Delta VL IgG)

### Method

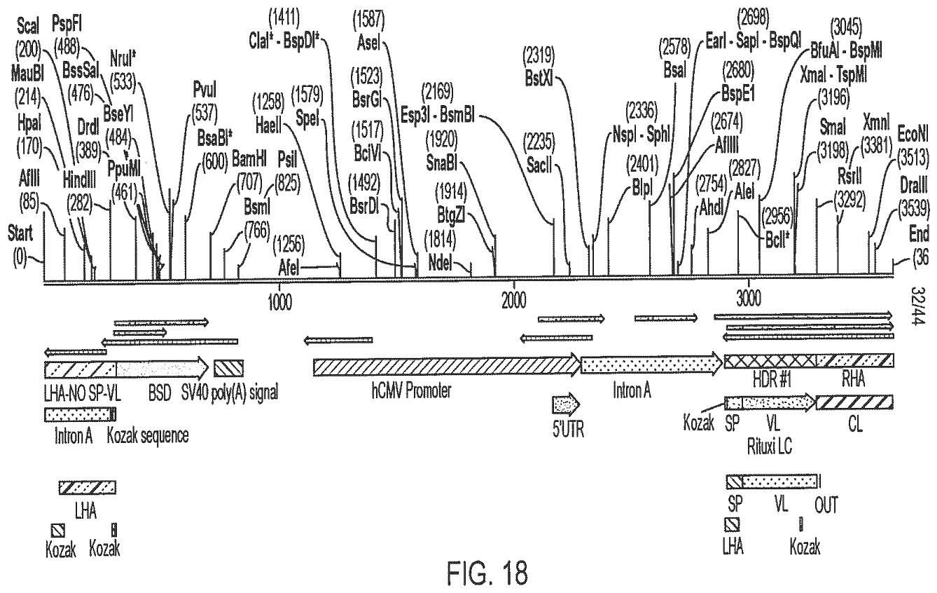

SSI cells expressing delta VL IgG antibody were sub-cultured in the same manner as before. sgRNA #9 was selected and prepared in the same manner as described before for nucleofection. 100 μg of light chain variable region DNA template was centrifuged briefly to collect contents and re-suspended with 27.7 μl of TE buffer to receive a concentration of 3.6 μg/μl. FIG. 18 shows representation of DNA template used for light chain variable region-Blasticidin integration.

Cas9 RNP complexes were prepared according to Table 12.

TABLE 12
RNP complexes

| Tube | Cas9 | sgRNA #9 | | DNA template | TE buffer | |
|---|---|---|---|---|---|---|
| No. | protein (μl) | (μl) | | (μg, μl) | (μl) | Nucleofection |
| 1 | 3.2 | 3.2 | Stable | 3.6 μg, 2 μl | 1.2 | + |
| | | | dsDNA | (out of 1.8 μg/μl) | | |
| | | | | (400 fmol per | | |
| | | | | reaction 3614 bp | | |
| | | | | 900 ng) | | |
| 2 | 3.2 | 3.2 | | 7.2 μg, 2 μl | 1.2 | + |
| | | | | (out of 3.6 μg/μl) | | |
| | | | | (800 fmol per | | |
| | | | | reaction3614 bp | | |
| | | | | 1.8 μg) | | |
| 3 | 3.2 | — | | 3.6 μg, 2 μl | 4.4 | + |
| | | | | (out of 1.8 μg/μl) | | |
| 4 | — | 3.2 | | 3.6 μg, 2 μl | 4.4 | + |
| | | | | (out of 1.8 μg/μl) | | |
| 5 | — | — | | — | 9.6 | + |
| 6 | 3.2 | 3.2 | | 3.6 μg, 2 μl | 1.2 | − |
| | | | | (out of 1.8 μg/μl) | | |
| 7 | — | — | | — | 9.6 | − |

SSI cells expressing VH only IgG antibody were prepared for nucleofection by transferring 8×10⁶ cells to a new tube and centrifuged at 200 g for 5 min to remove as much media as possible. The cells were then re-suspended in 563.2 μl of Nucleofection solution (600 μl of SF solution prepared by mixing 492 μl of SF solution with 108 μl of supplement 1). 70.4 μl of cells were added to each of the Cas9 RNP complex mixtures.

20 μl of the cells-nucleofection solution-RNP complex mixture were transferred to a cuvette in the nucleofection 16-well strip where cells were pulsed using the Lonza 4D nucleofector unit using program DU-158. The cells were left to sit in the nucleofection cuvettes for 10 min post nucleofection and then added and mixed 80 μl of pre-warmed 10 ml of CD-CHO medium warmed to a 36.5° C. The entire 100 μl of the final mixtures were transferred to the 12-well plate with 1.5 ml of a CD-CHO medium, kept in the incubator at 36.5° C. The cells were then incubated at 36.5° C., 5% CO₂ in a static incubator for 24 hours.

24 hours later, medium was replaced with CD-CHO+6 μg/mL Puro+5 μg/mL Blasticidin medium for selection. Cells were grown in static incubator until negative control were died. Cell viability assessment was performed using Vi-Cell device.

### Results

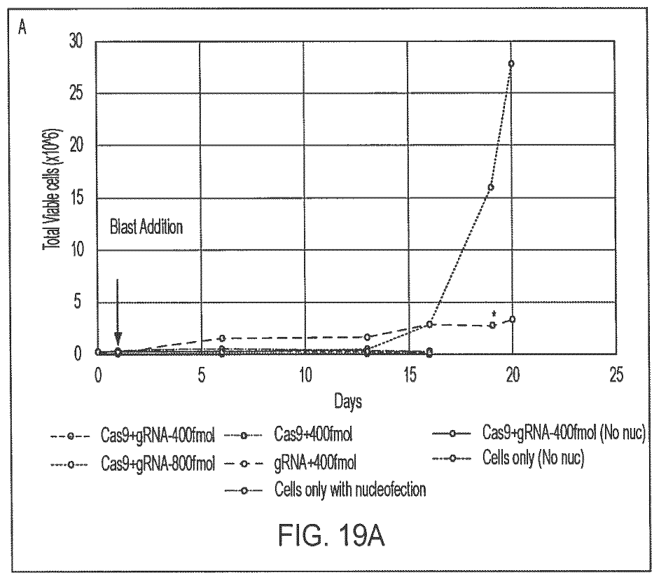

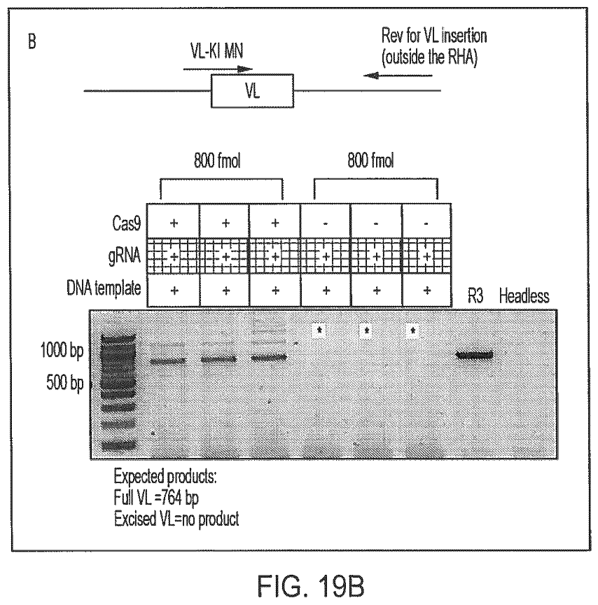

Integration of light chain variable region validated by PCR. Cell selection and viability assessed through Blasticidin selection as shown in FIG. 19A. PCR evaluation (FIG. 19B) shows seamless integration of light chain variable region as indicated by the bands at the 764 bp in the left 3 columns.

### Example 15: Creating Inducible Cas9 Single Site Integration BOB (iBOB) Cells

### Method

Transduction medium was prepared by diluting Polybrene from stock solution (10 mg/mL) to a final concentration of 10 μg/mL (1:1000 dilution factor), which was divided into Tube 1 and Tube 2 with 6 mL and 3 mL, respectively. Then Edit-R inducible lentiviral hEF1a-Cas9 nuclease particles were added to Tube 2. The volume of lentiviral particles to be added was determined by the equation:

$$V = M O I \times CN \div VT \times 1000$$

where:
   V=volume of lentiviral stock in μL MOI=desired multiplicity of infection
   CN=number of cells in the well at transduction
   VT=lentiviral titer in TU/mL and multiplied by 1000 to convert the volume from mL to μL.

Based on the desired MOI of 1, cell density of 0.1×10⁶ cells per well at the time of transduction, and Lentiviral titer of 1×10⁷ TU/mL, 25 μL of lentiviral stock per well was calculated.

BOB cells were prepared in Tube A and Tube B at the amount of 0.6×10⁶ cells and 0.3×10⁶, respectively, and centrifuged at 200 g for 5 minutes. The cells were then resuspended with the transduction medium as prepared above so that the content of Tube 1 is added to Tube A to contain cells without lentiviral particles and Tube 2 is added to Tube B to contain cells with lentiviral particles. These cells were seeded into a 12-well plate according to plate layout of Table 13 with 1 ml in each well and incubated at 37° C. in a humidified CO₂ incubator for 24 hours.

TABLE 13
Plate layout for cell seeding

| | | |
|---|---|---|
| Well #1 | Well #4 | Well #7 |
| Tube 1 | Tube 1 | Tube 2 |
| (Untransduced) | (Untransduced + Blast) | Blast + LV) |
| Well #2 | Well #5 | Well #8 |
| Tube 1 | Tube 1 | Tube 2 |
| (Untransduced) | (Untransduced + Blast) | Blast + LV) |
| Well #3 | Well #6 | Well #9 |
| Tube 1 | Tube 1 | Tube 2 |
| (Untransduced) | (Untransduced + Blast) | Blast + LV) |

After 24 hours, the content of each well was centrifuged at 200 g for 5 minutes to remove the medium, and resuspended with 1 ml of pre-warmed CD-CHO/6 mM L-glutamine medium, prepared by adding 10 μL Polybrene to 10 mL CD-CHO/6 mM L-Glutamine, and seeded in a new plate according to the plate layout of Table 4 with 1 ml in each well. The cells were incubated at 37° C. in a humidified CO₂ incubator for 72 hours.

After 72 hours, selection medium containing Blasticidine in a concentration of 5 μg/mL were used to select for transduced cells. The cells were subcultured as needed and Blasticidine was added to appropriate wells at each subculturing.

### Results

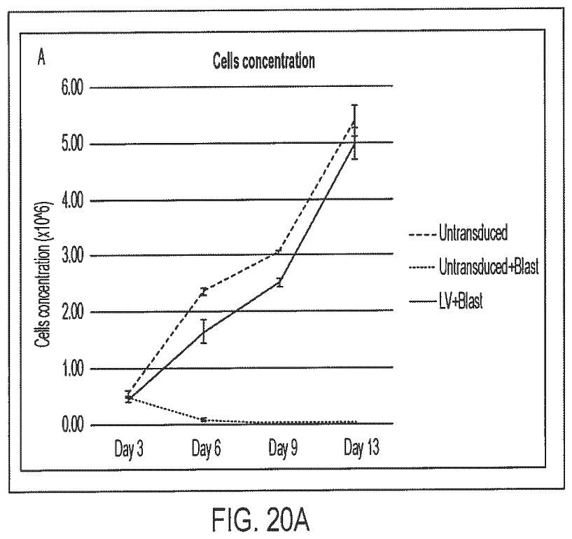

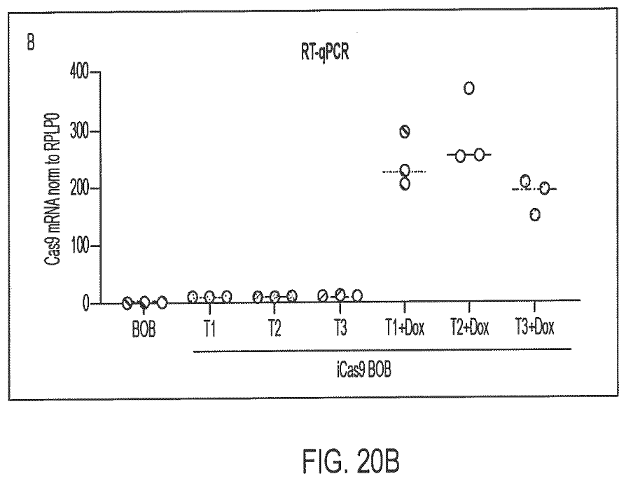

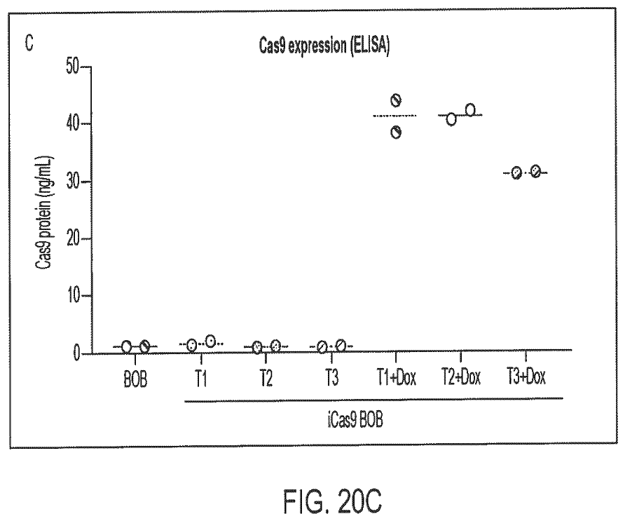

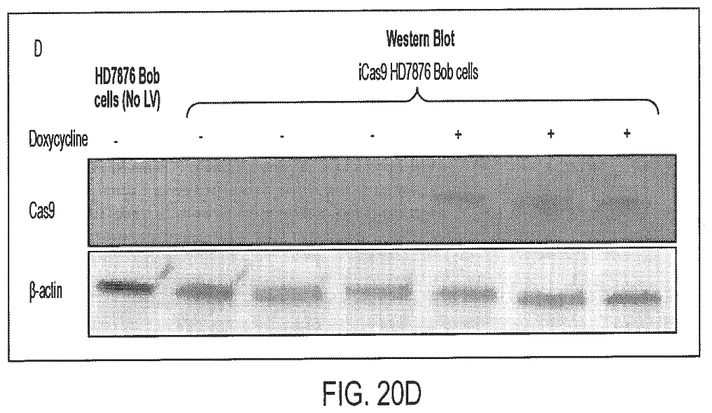

Cell viability assessment shows successful transduction, selection, proliferation, and cell banking of inducible-Cas9 BOB (SSI) cell line as shown in FIG. 20A. RT-PCR, ELISA, and WB analysis also validate generation of inducible Cas9 BOB (iBOB) cell line and evaluate Cas9 expression, as shown in FIGS. 20B-20D.

### Example 16: Creating Inducible Cas-SSI Cell Line Expressing IgG Antibody Lacking its VL Domain

### Method

iBOB host cells were sub-cultured at a viable concentration of 0.3×10⁶ cells/mL into an appropriately sized Erlenmeyer shake-flask (with CD-CHO medium supplemented with 6 mM L-glutamine). These cells have been in culture for no less than 4 days and no more than 4 weeks and have viability greater than 90% prior to transfection.

Purified delta VL plasmid containing the gene of interest and pMF26 plasmid containing an FlpE recombinase expression cassette were mixed in a DNase free sterile tube and the volume adjusted to 100 μL using TE buffer, according to Table 14.

TABLE 14
Plasmid mixture preparation

| | pEE_QGV_SSI | pMF26 | | |
|---|---|---|---|---|
| | (5 × 4 μg) | (45 μg per | TE buffer | |
| | (5 μg per 1 replica) | 1 replica) | (μl) | Electroporation |
| 1 | Delta VL (10 μg) | + (90 μg) | Up to 200 μl | + |
| 2 | Delta VL (5 μg) | + (45 μg) | Up to 100 μl | − |
| 3 | Delta VL (5 μg) | − | Up to 100 μl | + |
| 4 | — | − | 100 μl | + |
| 5 | — | − | 100 μl | − |

100 μL of plasmid mix from Table 6 were transferred to a 0.4 cm electroporation cuvette (per transfection), to which 0.8 ml of iBOB host cells, prepared and resuspended in 6.4 ml CD CHO/6 mM L-glutamin medium, were added. The cells were electroporated by delivering a single exponential decay pulse of 300 V, 900 μF.

The contents of the cuvette were transferred to each of the 20 mL of CD CHO/6 mM L-glutamine medium added to six T75 flasks and pre-warmed to 36.5° C. in a humidified static CO₂ incubator. The flasks were incubated for 24 hours in the humidified static CO₂ incubator.

After 24 hours, the medium of each flask was replaced with 20 ml pre-warmed CD CHO medium. The cells were cultured and subcultured.

### Results

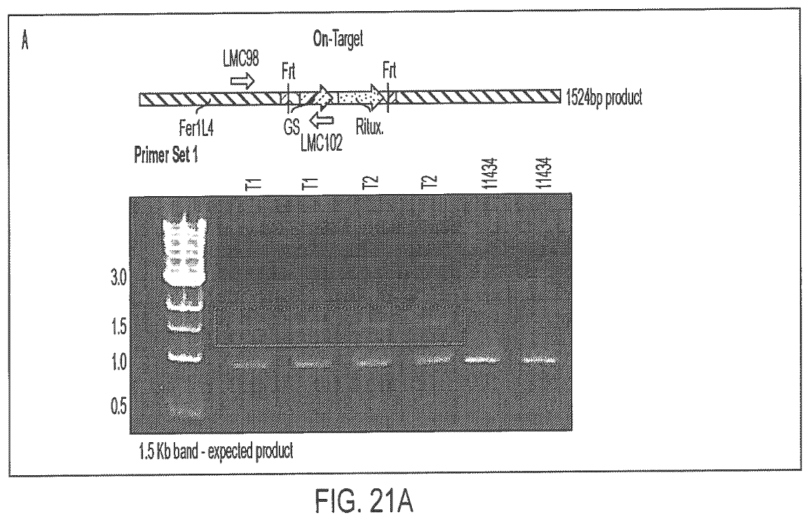

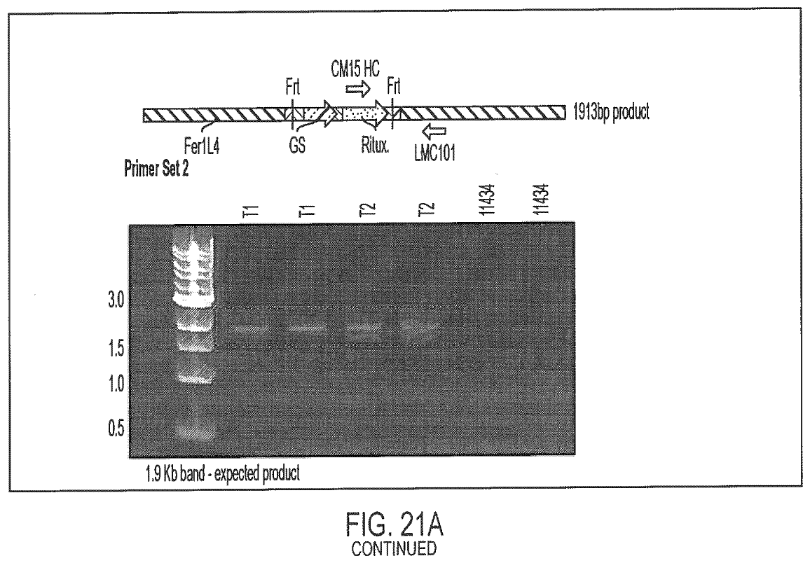

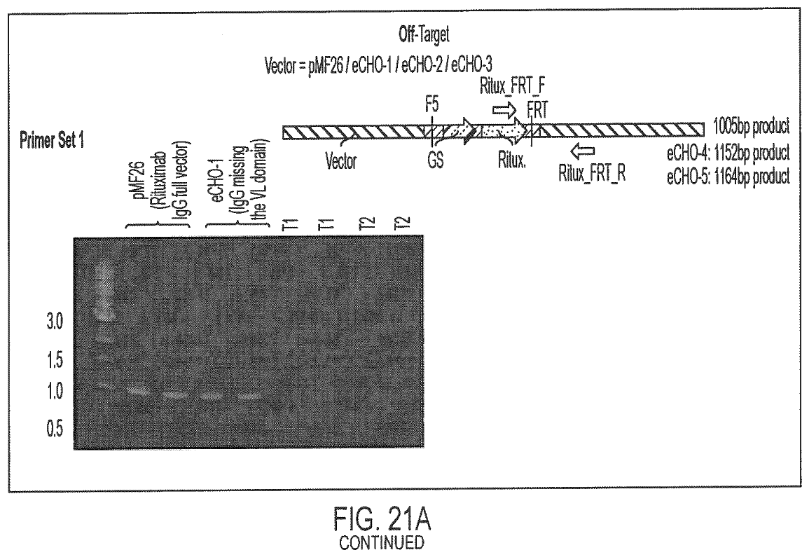

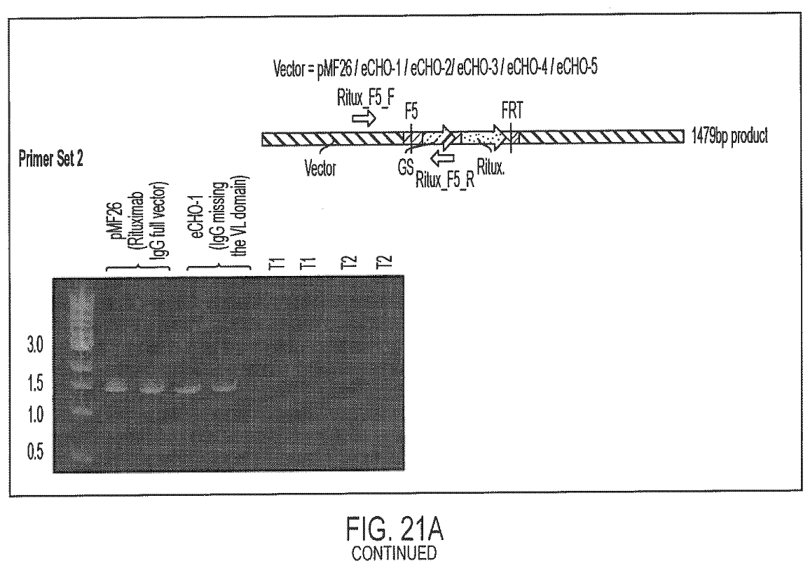

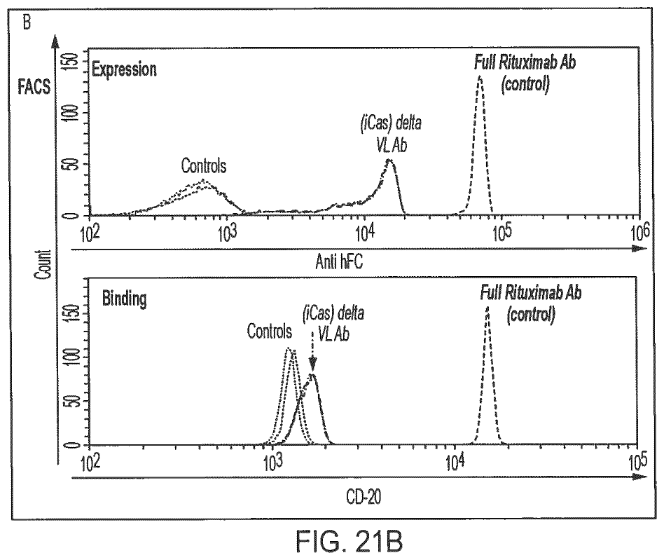

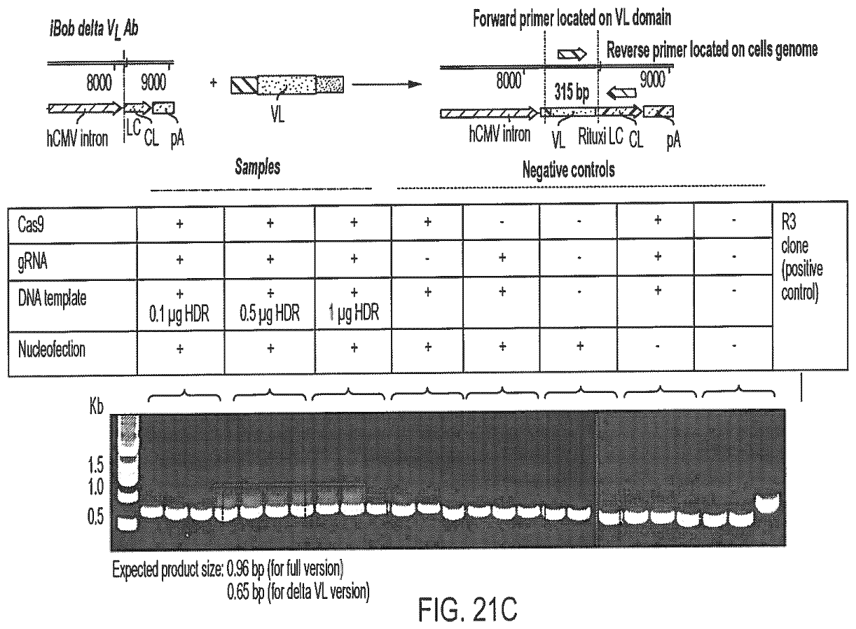

PCR and FACS analysis as shown in FIGS. 21A and 21B validate successful generation of delta VL iBOB cells.

### Example 17: Creating Inducible Cas-SSI Cell Line Expressing IgG Antibody Lacking its VH Domain

### Method

iCas9 CHO host cells were sub-cultured at a viable concentration of 0.3×10⁶ cells/mL into an appropriately sized Erlenmeyer shake-flask (with CD-CHO medium supplemented with 6 mM L-glutamine). These cells have been in culture for no less than 4 days and no more than 4 weeks and have viability greater than 90% prior to transfection.

Purified delta VH plasmid containing the gene of interest and pMF35 plasmid containing an FlpE recombinase expression cassette were mixed in a DNase free sterile tube and the volume adjusted to 100 μL using TE buffer, according to Table 15.

TABLE 15
Plasmid mixture preparation

| | pEE_QGV_SSI | pMF35 | | |
|---|---|---|---|---|
| | (5 × 4 μg) | (45 μg per | TE buffer | |
| | (5 μg per 1 replica) | 1 replica) | (μl) | Electroporation |
| 1 | Delta VH (10 μg) | + (90 μg) | Up to 200 μl | + |
| 2 | Delta VH (5 μg) | + (45 μg) | Up to 100 μl | − |
| 3 | Delta VH (5 μg) | − | Up to 100 μl | + |
| 4 | — | − | 100 μl | + |
| 5 | — | − | 100 μl | − |

100 μL of plasmid mix from Table 10 were transferred to a 0.4 cm electroporation cuvette (per transfection), to which 0.8 ml of iCas CHO host cells, prepared and resuspended in 6.4 ml CD CHO/6 mM L-glutamin medium, were added. The cells were electroporated by delivering a single exponential decay pulse of 300 V, 900 μF.

The contents of the cuvette were transferred to each of the 20 mL of CD CHO/6 mM L-glutamine medium added to six T75 flasks and pre-warmed to 36.5° C. in a humidified static CO₂ incubator. The flasks were incubated for 24 hours in the humidified static CO₂ incubator.

After 24 hours, the medium of each flask was replaced with 20 ml pre-warmed CD CHO medium. The cells were cultured and subcultured.

1-28. (canceled)

29. A method of producing an editable Chinese Hamster Ovary (CHO) cell, comprising:
a) providing a CHO cell stably expressing a genomic nucleic acid sequence of an antibody that includes a variable heavy chain region sequence, constant heavy chain regions 1, 2 and 3 sequences, a variable light chain region sequence, and constant light chain region 1 sequence;
b) excising the sequence encoding the variable heavy chain region with a gene editing protein, wherein excising the sequence encoding the variable heavy chain region with the gene editing protein occurs at a first guide RNA target sequence and a second guide RNA target sequence;
c) introducing a first guide RNA and a second guide RNA, wherein:
i. the first guide RNA has the amino acid sequence shown in SEQ ID NO: 34 and the second guide RNA has the amino acid sequence shown in SEQ ID NO: 36; or
ii. the first guide RNA has the amino acid sequence shown in SEQ ID NO: 36 and the second guide RNA has the amino acid sequence shown in SEQ ID NO: 34;
d) excising the sequence encoding the variable light chain region sequence with the gene editing protein, wherein excising the variable light chain region sequence with the gene editing protein occurs at a third guide RNA target sequence and a fourth guide RNA target sequence; and
e) introducing a third guide RNA and a fourth guide RNA, wherein:
i. the third guide RNA has the amino acid sequence shown in SEQ ID NO: 1 and the fourth guide RNA has the amino acid sequence shown in SEQ ID NO: 7; or
ii. the third guide RNA has the amino acid sequence shown in SEQ ID NO: 7 and the fourth guide RNA has the amino acid sequence shown in SEQ ID NO: 1.

30. The method of claim 29, further comprising:
a) introducing a sequence encoding the gene editing protein to the genomic nucleic acid sequence prior to the excising the sequence encoding the variable heavy chain region and the variable light chain region; and
b) expressing the gene editing protein prior to the excising the variable heavy chain region and the variable light chain region.

31. The method of claim 29 or 30, wherein the gene editing protein is a Cas gene editing protein.

32. The method of claim 29 or 30, wherein the gene editing protein is selected from Cas9, Cas12, Cas1212 TALENS, MAD7 nuclease and a Zinc Finger Nuclease.

33. The method of claim 32, wherein the gene editing protein is Cas 9.

34. The method of any one of claims 29-33, wherein the sequence encoding the gene editing protein is operably connected to an inducible promoter.

35. The method of claim 34, wherein the inducible promoter is a TET-on system.

36. The method of any one of claims 29-35, wherein the editable cell is a high expressing, stable clone.

37. A method of making an antibody producing Chinese Hamster Ovary (CHO) cell, comprising:
producing an editable Chinese Hamster Ovary (CHO) cell using the method of any one of claims 29-36;
introducing a sequence encoding an antibody heavy chain variable region to the cell; and
introducing a sequence encoding an antibody light chain variable region to the cell.

38. The method of claim 37, further comprising introducing a fifth guide RNA target sequence and a sixth guide RNA target sequence.

39. The method of claim 38, wherein:
a) the fifth guide RNA has the amino acid sequence shown in SEQ ID NO: 39 and the sixth guide RNA has the amino acid sequence shown in SEQ ID NO:6; or
b) the first guide RNA has the amino acid sequence shown in SEQ ID NO: 6 and the second guide RNA has the amino acid sequence shown in SEQ ID NO:39.

40. The method of any one of claims 37-39, further comprising introducing a first sequence encoding a first selectable marker and a second sequence encoding a second selectable marker.

41. The method of any one of claims 37-40, further comprising selecting a cell expressing the antibody using the first and the second selectable markers.

42. The method of any one of claims 37-41, further comprising expressing the antibody in the cell.
